(19) United States
(12) Patent Application Publication
(10) Pub. No.: US 2020/0270322 A1
(43) Pub. Date: Aug. 27, 2020

(54) NOVEL INTERLEUKIN 4 IMMUNOCONJUGATES

(71) Applicant: PHILOGEN S.P.A., SIENA (IT)

(72) Inventors: Mattia Matasci, Zurich (CH); Francesca Pretto, Zurich (CH); Sarah Wulhfard, Baden (CH)

(21) Appl. No.: 16/341,644

(22) PCT Filed: Oct. 12, 2017
(86) PCT No.: PCT/EP2017/076116

§ 371 Date: Apr. 12, 2019

(30) Foreign Application Priority Data
Oct. 14, 2016 (GB) 1617453.4
Aug. 9, 2017 (GB) 1712755.6

(51) Int. Cl. C07K 14/54; C07K 14/55; C07K 14/705; C07K 16/24; C07K 16/40; A61K 47/68; A61P 35/00

(52) U.S. Cl. CPC C07K 14/5406; C07K 14/5428; C07K 14/5434; C07K 14/55; A61P 35/00; C07K 16/247; C07K 16/40; A61K 47/6813; A61K 47/6851; C07K 14/70578

(57) ABSTRACT

This invention relates to an immunoconjugate comprising interleukin-4 (IL4) and two antibody molecules which bind an extra-cellular matrix component associated with neoplastic growth, angiogenesis, and/or tissue remodelling, such as the A1 domain of tenascin C or the ED-A or ED-B isoforms of fibronectin. The antibody molecules may be positioned at the N and C terminals of the IL4. The immunoconjugate may be useful in the treatment of cancer, inflammatory autoimmune disease and other disease characterised by expression of an extra-cellular matrix component associated with neoplastic growth, angiogenesis, and/or tissue remodelling.

### FIELD

The present invention relates to conjugates comprising a cytokine and two specific binding members. The specific binding members preferably bind an extra-cellular matrix component associated with neoplastic growth and/or angiogenesis, and the conjugate may be used, for example, for targeting cytokine to tissues in vivo. In particular, the present invention relates to the therapeutic use of such conjugates in which cytokine is conjugated to two specific binding members through a peptide bond at the C terminus and the N terminus of the cytokine molecule.

### BACKGROUND

Cytokines are key mediators of innate and adaptive immunity. Many cytokines have been used for therapeutic purposes in patients, such as those with advanced cancer, but their administration is typically associated with severe toxicity, hampering dose escalation to therapeutically active regimens and their development as anticancer drugs, for example. To overcome these problems, the use of ‘immunocytokines’ (i.e. cytokines fused to antibodies or antibody fragments) has been proposed, with the aim of concentrating the immune-system stimulating activity at the site of disease, whilst sparing normal tissues (Savage et al., 1993; Schrama et al., 2006; Neri et al. 2005; Dela Cruz et al., 2004; Reisfeld et al., 1997; Konterman et al., 2012).

Several pro-inflammatory immunocytokines (e.g., those based on IL2, IL12, IL15, TNF) have been shown to display a potent anti-tumoural effect in mouse models of cancer (Borsi et al. 2003; Carnemolla et al., 2002; Frey et al., 2010; Kaspar et al., 2007; Pasche et al., 2012). In contrast, anti-inflammatory immunocytokines (e.g., those based on IL10) have been shown to confer a therapeutic benefit in mouse models of chronic inflammatory conditions (rheumatoid arthritis, endometriosis (Schwager et al. 2011; Schwager et al., 2009)) but to have no impact on tumour growth.

Antibodies specific to splice-isoforms of fibronectin and of tenascin-C have been described as vehicles for pharmacodelivery applications, as these antigens are virtually undetectable in the normal healthy adult (with the exception of the placenta, endometrium and some vessels in the ovaries) while being strongly expressed in the majority of solid tumours and lymphomas, as well as other diseases (Brack et al., 2006; Pedretti et al., 2009; Schliemann et al. 2009). For example, antibodies F8 and L19, specific to the alternatively-spliced EDA and EDB domains of fibronectin, respectively, and anti-tenascin C antibody F16 (Brack et al. 2006, Villa et al., 2008, Viti et al., 1999), have been employed for the development of armed antibodies, some of which have begun clinical testing in oncology and in rheumatology (Eigentler et al., 2011; Papadia et al., 2012). The tumour targeting properties of these antibodies have also been documented in mouse models of cancer and in patients.

Interleukin 4 (IL4) is a 14 kDa compact globular cytokine, stabilized by three internal disulphide bonds. It was first identified in the early 1980s as a B cell activating factor and exhibits many biological and immunoregulatory functions. It can control proliferation, differentiation and apoptosis in several cell types of hematopoietic and non-hematopoietic origin, including myeloid, mast, dendritic, endothelial, muscular and neuronal cells (Janeway, Immunobiology, 2005; Zamorano et al., 1996). As a key regulator in humoral and adaptive immunity, IL4 acts as a growth and survival factor for lymphocytes, stimulating the proliferation of activated B cells and T cells. The cytokine is crucially involved in the balance between Th1 and Th2 immunological responses, inducing the differentiation of naïve helper T cells into Th2 cells after antigen challenge (Janeway, Immunobiology, 2005). This activity is in stark contrast to the activity of IL12, which drives a Th1 polarization of immune response. Interleukin 4 also stimulates the proliferation of NK (natural killer) cells and up-regulates MHC class II production, therefore enhancing the antigen presentation (Chomarat et al., 1997).

Nowadays, IL4 is mostly considered to be an anti-inflammatory cytokine. However, although IL4 has been shown to exhibit disease-suppressing effects in in vivo mouse models of collagen-induced arthritis when high doses of murine IL4 were administered (Joosten et al., 1999), administration of low doses of murine IL4 had no effect on the course of arthritis in the same mouse model (Joosten et al., 1999; Joosten et al., 1997). In contrast, administration of even low doses of murine IL10 in this mouse model led to suppression of arthritis (Joosten et al., 1997). In addition, IL4 does not exhibit anti-inflammatory properties under all conditions. For example, IL4 treatment significantly accelerates the development of colitis in a mouse model of the disease (Fort et al., 2001). IL10 therefore represents a more promising candidate than IL4 for the preparation of immunoconjugates, in particular for the treatment of inflammatory conditions, such as RA and colitis.

The effect of IL4 on tumours is also far from clear. It appears that both expression patterns and doses influence the effect of IL4 on tumour growth. For example, opposite biological effects on endothelial cell migration have been observed at low (promotion) and high concentrations (inhibition) of IL4 (Volpert et al., 1998). Li et al. (2008) reports that endogenous IL4 promotes tumour development by increasing tumour cell resistance to apoptosis while exogenous IL4 has anti-tumour effects. Fukushi et al. (2000) similarly discusses the variable effects of IL4 on angiogenesis reported in the literature, with the authors themselves concluding that IL4 induces angiogenesis both in vitro and in vivo in a corneal pocket assay. The paradoxical effect of IL4 on tumours is summarised, for example, in Li et al. (2009).

Although preclinical studies with recombinant, untargeted, murine IL4 as therapeutic agent showed promising anti-tumour activity in various mouse models of cancer (Tepper et al., 1989; Tepper et al., 1992; Wei et al., 1995; Yu et al., 1993), leading to the clinical investigation of recombinant human IL4 in several cancer types (Wiernik et al., 2010; Whitehead et al., 2002), only minimal anti-tumour activity was reported in several clinical studies with more than 154 patients. Only one complete response in a patient with disseminated malignant melanoma and one in a patient with relapsed and resistant NHL was observed. Furthermore, IL4 therapy had substantial toxicity, the most common side effects being nausea, vomiting, diarrhoea, headache/pain or malaise/fatigue/lethargy, including cases of grade 4 toxicities. As a consequence, the systemic use of IL4 was determined to be unsuitable for cancer treatment (Whitehead et al., 2002; Whitehead et al., 1998; Kurtz et al., 2007).

Cytokines can be conjugated to antibody molecules to produce immunocytokines as mentioned above. However, although several immunocytokines have been successfully made and tested, not all immunocytokines exhibit therapeutic effects, even when such therapeutic effects would be expected from the effects of treatment with the untargeted cytokine. For example, F8-IL7, F8-IL17, F8-IFN-alpha and IFN-gamma did not display the expected therapeutic effects or pharmaceutical quality when tested in mice (Pasche et al., 2011; Pasche et al., 2012; Frey et al., 2011; Ebbinghaus et al., 2005). In addition, not all immunocytokines retain the in vivo targeting properties of the parental antibody (Pasche & Neri 2012). Even when the targeting properties of the parental antibody are retained and the immunocytokine localizes efficiently to the site of disease, the therapeutic effect of the immunocytokine may be no better than that of the untargeted immunocytokine (Frey et al., 2011). The preparation of immunocytokines with therapeutic effects, such as anti-tumoural or anti-inflammatory activity, in particular the preparation of immunocytokines with more potent therapeutic activity than the untargeted cytokine, is therefore far from straightforward. The preparation of a conjugate comprising IL4 fused to the Fc region of murine IgG2a is described in Walz et al. (2002). The authors postulate that fusion of the Fc region to IL4 will result in an increase the half-life of the conjugate compared with monomeric IL4, although no evidence for this is provided. The purpose of the Fc region in this conjugate is therefore not to target the IL4 to regions of disease as is the case with immunocytokines as described above.

The possibility of conjugating IL4 to targeting moieties is mentioned in WO2003/092737 and WO2001/10912. However, a conjugate comprising IL4 and a specific binding member which binds an extra-cellular matrix component associated with neoplastic growth and/or angiogenesis is not disclosed in either of these documents, nor is any therapeutic effect for such a conjugate demonstrated.

WO2014/173570 reports that IL4 can be successfully conjugated to a diabody specific for the EDA domain of fibronectin termed “F8” and successfully treat serious disorders in animal models, including endometriosis, multiple sclerosis and psoriasis. WO2014/173570 also shows the successful targeting of tumors when murine IL4 was conjugated to the same anti-EDA diabody F8.

### SUMMARY

The present inventors have shown that immunocytokines comprising human interleukin-4 (IL4) conjugated to two specific binding members retain both the targeting properties of the unconjugated specific binding members and the biological activity of IL4.

An aspect of the invention provides a conjugate comprising interleukin-4 (IL4) and two specific binding members.

The two specific binding members preferably bind an extra-cellular matrix component associated with neoplastic growth, angiogenesis, and/or tissue remodelling, most preferably an extra-cellular matrix component associated with neoplastic growth and/or angiogenesis, such as the Extra Domain-A (ED-A) isoform of fibronectin, the Extra Domain-B (ED-B) isoform of fibronectin, or tenascin C.

Other aspects of the invention are described below.

### BRIEF DESCRIPTION OF THE FIGURES

FIG. 1 shows a schematic representation of the different molecular formats of human IL4 fusions.

FIG. 1(A) shows IL4-DbF8 in which IL4 is conjugated via its C terminus to the F8 antibody in diabody configuration.

FIG. 1(B) shows the “old format” of DbF8-IL4 disclosed in WO2014/173570 in which IL4 is conjugated via its N terminus to the F8 antibody in diabody configuration.

FIG. 1(C) shows a first new format (IL4-ScDbF8) in which IL4 is conjugated via its C terminus to two F8 antibodies expressed in scDb configuration.

FIG. 1(D) shows a second new format (ScDbF8-IL4) in which IL4 is conjugated via its N terminus to two F8 antibodies expressed in scDb configuration.

FIG. 1(E) shows the third and preferred format (ScFvF8-IL4-ScFvF8) which IL4 is conjugated via its N terminus to a first F8 antibody expressed in scFv configuration and via its C terminus to a second F8 antibody expressed in scFv configuration.

FIG. 2 shows the biochemical properties of purified conjugates.

FIG. 2(A) shows SDS-PAGE analysis of hIL4-ScDbF8, ScDbF8-hIL4, and ScFvF8-IL4-ScFvF8, as visualised by Coomassie staining.

FIG. 2(B) shows size exclusion chromatography analysis of IL4-DbF8, DbF8-IL4, and ScFvF8-IL4-ScFvF8.

FIG. 3 shows the activity of purified conjugates.

FIG. 3(A) shows surface plasmon resonance (Biacore) analysis of the binding capacity of the F8 moiety to the ED-A of fibronectin.

FIG. 3(B) shows ELISA analysis of the binding capacity of the F8 moiety to the ED-A of fibronectin.

FIG. 3(C) shows the results of a TF-1 proliferation assay to determine the biological activity of human IL4 after fusion with the F8 antibody in the ScDbF8-IL4, ScFvF8-IL4-ScFvF8 and IL4-ScDbF8 fusion proteins FIG. 4 shows the pharmacokinetic (PK) profiles of DbF8-hIL4 and the novel format ScFvF8-hIL4-ScFvF8 following a single bolus injection in cynomolgus monkeys.

FIG. 5 shows a comparative study of the tumor targeting performance of the ScFvF8-mIL4-ScFvF8 and ScFvF8-mIL7-ScFvF8 in BALB/c nude mice bearing subcutaneously implanted A431 human squamous carcinoma. Mice were sacrificed after 24 h, organs were weighed and radioactivity counted, expressing results as percentage of injected dose per gram of tissue (% ID/g).

### DETAILED DESCRIPTION

This invention relates to immunoconjugates comprising interleukin-4 (IL4) and two specific binding members. The two specific binding members bind to the same extra-cellular matrix component. Immunoconjugates are shown herein to retain both the targeting properties of the unconjugated specific binding members and the biological activity of IL4.

The preparation of immunoconjugates is difficult and unpredictable and there is no guarantee of success with any molecular format. The prior art reports only few teachings on the molecular formats of immunocytokines. For example, in WO2013/010749, the cytokine IL12 is conjugated via its N-terminus and C-terminus to two single-chain antibodies the tumor targeting ability of F8-IL12-F8 shown to be inferior to IL12 conjugated to a single-chain diabody (IL12-F8-F8). Pasche et al. (2011) report that when the cytokine IL7 is conjugated via its N-terminus and C-terminus to two single-chain antibodies (F8-IL7-F8) “only a subtle superiority” could be observed when a tumor targeting antibody moiety (F8-mIL7-F8) was used instead of an antibody of irrelevant specificity” (page 91 first paragraph). In view of these attempts, the superior tumor targeting profiles of IL4 immunoconjugates of the invention compared to IL4 fused to a single chain diabody, are entirely unexpected.

The specific binding members in the conjugates described herein are preferably antibody molecules, such as antibodies or antigen binding fragments thereof. In some preferred embodiments, the antibody molecules may comprise or consist of single chain Fvs (ScFvs), diabodies or single chain diabodies. Most preferably, the antibody molecules are single chain Fvs (ScFvs).

In some embodiments exemplified herein, a first ScFv antibody may be conjugated at the N terminus of human IL4 and a second scFv antibody with the same specificity, may be conjugated at the C terminus of human IL4 (i.e. ScFv-IL4-ScFv). The resultant immunoconjugate shows a superior targeting performance as compared to:
(i) a single chain diabody with same specificity, sequentially conjugated at the N terminus of human IL4 (i.e. ScDb-IL4), or
(ii) a single chain diabody with same specificity, sequentially conjugated at the C terminus of human IL4 (IL4-ScDb), or
(iii) a diabody with same specificity, sequentially conjugated at the N terminus of human IL4 (Db-IL4), or
(iv) a diabody with same specificity, sequentially conjugated at the C terminus of human IL4 (IL4-Db).

“Single-chain Fv” or “ScFv” antibody fragments comprise the VH and VL domains of antibody, wherein these domains are present in a single polypeptide chain. Preferably, the Fv polypeptide further comprises a polypeptide linker between the VH and VL domains which enables the ScFv to form the desired structure for antigen binding. For a review of ScFv see Pluckthun in The Pharmacology of Monoclonal Antibodies, vol. 113, Rosenburg and Moore eds., Springer-Verlag, New York, pp. 269-3 15 (1994). The polypeptide linker between the VH and VL domains usually consists of at least 10 amino acids.

Where the antibody molecule is an scFv, the VH and VL domains of the antibody are preferably linked by a 10 to 20 amino acid linker. For example, the VH and VL domains may be linked by an amino acid linker which is 10, 11, 12, 13, 14, 15, 16, 17, 18, 19, or 20 amino acid in length. Suitable linker sequences are known in the art and include the linker sequences set forth in SEQ ID NO: 40, SEQ ID NO: 44, or SEQ ID NO: 45.

Diabodies are multimers of polypeptides, each polypeptide comprising a first domain comprising a binding region of an immunoglobulin light chain and a second domain comprising a binding region of an immunoglobulin heavy chain, the two domains being linked (e.g. by a peptide linker) but unable to associate with each other to form an antigen binding site: antigen binding sites are formed by the association of the first domain of one polypeptide within the multimer with the second domain of another polypeptide within the multimer (WO1994/13804; Holliger and Winter, Cancer Immunol. Immunother. (1997) 45:128-130; Holliger et al., Proc. Natl. Acad. Sci. USA 90 6444-6448, 1993).

In a diabody (“Db”) a heavy chain variable domain (VH) is connected to a light chain variable domain (VL) on the same polypeptide chain. The VH and VL domains are connected by a peptide linker that is too short to allow pairing between the two domains (generally around 5 amino acids). This forces paring with the complementary VH and VL domains of another chain. The VH and VL domains in a diabody are thus preferably linked by a 5 amino acid linker. The linker preferably has the sequence shown in SEQ ID NO: 39. The linker may consist of 3, 4, 5 or 6 amino acids.

Alternatively, a diabody may be a single chain diabody (“ScDb”). In a single chain diabody two sets of VH and VL domains are connected together in sequence on the same polypeptide chain. For example, the two sets of VH and VL domains may be assembled in a single chain sequence as follows: (VH-VL)-(VH-VL), where the brackets indicate a set. In the single chain diabody format each of the VH and VL domains within a set is connected by a short or ‘non-flexible’ peptide linker. This type of peptide linker sequence is not long enough to allow pairing of the VH and VL domains within the set. Generally, a short or ‘non flexible’ peptide linker is around 5 amino acids. The two sets of VH and VL domains are connected as a single chain by a long or ‘flexible’ peptide linker. This type of peptide linker sequence is long enough to allow pairing of the VH and VL domains of the first set with the complementary VH and VL domains of the second set. Generally, a long or ‘flexible’ linker is around 15 amino acids. Single chain diabodies have been previously generated (Kontermann, R. E., and Muller, R. (1999), J. Immunol. Methods 226: 179-188). A bispecific single chain diabody has been used to target adenovirus to endothelial cells (Nettelbeck et al., Molecular Therapy (2001) 3, 882-891).

The specific binding members preferably bind an extra-cellular matrix (ECM) component associated with neoplastic growth and/or angiogenesis, as mentioned above. The specific binding members may bind fibronectin. For example, the specific binding members may bind the Extra Domain-A (ED-A) isoform or Extra Domain-B (ED-B) isoform of fibronectin, or tenascin C. Preferably, the specific binding members bind the ED-A or ED-B isoform of fibronectin, or bind the A1 domain of tenascin C. Most preferably, the specific binding members bind the ED-A isoform of fibronectin.

Preferably, the specific binding members in the conjugates of the invention have the same specificity (i.e. the conjugate is monospecific) and bind to the same extra-cellular matrix (ECM) component. For example, the conjugates may comprise two copies of the same specific binding member.

The specific binding members may comprise an antigen binding site having the complementarity determining regions (CDRs), or the VH and/or VL domains of an antibody capable of specifically binding to an antigen of interest, for example, one or more CDRs or VH and/or VL domains of an antibody capable of specifically binding to an antigen of the ECM. Such antigens include fibronectin and tenascin C, as described above.

Thus, the specific binding members may comprise an antigen binding site of the antibody F8, the antibody L19 or the antibody F16, which have all been shown to bind specifically to ECM antigens. The specific binding members may comprise an antigen binding site having one, two, three, four, five or six CDRs, or the VH and/or VL domains of antibody F8, L19 or F16. The specific binding members may comprise or consist of the sequence of antibody F8, L19 or F16, in scFv format.

F8, as referred to herein, is a human monoclonal antibody to the alternatively spliced ED-A domain of fibronectin. The VH sequence of the F8 antibody is shown in SEQ ID NO: 1. The VL sequence of the F8 antibody is shown in SEQ ID NO: 2. A ScFv version of this antibody is described in Villa A et al. (Int. J. Cancer. 2008 Jun. 1; 122(11): 2405-13) and in WO2008/120101. The sequence of F8 (ScFv) is shown in SEQ ID NO: 3. The sequence of F8 (Db) is shown in SEQ ID NO: 4.

An antigen binding site may comprise one, two, three, four, five or six CDRs of antibody F8. Amino acid sequences of the CDRs of F8 are:
SEQ ID NO:5 (CDR1 VH);
SEQ ID NO:6 (CDR2 VH);
SEQ ID NO:7 (CDR3 VH);
SEQ ID NO:8 (CDR1 VL);
SEQ ID NO:9 (CDR2 VL), and/or
SEQ ID NO:10 (CDR3 VL).

L19 is a human monoclonal antibody specific to the alternatively spliced ED-B domain of fibronectin and has been previously described (WO2006/119897). The VH sequence of the L19 antibody is shown in SEQ ID NO: 11. The VL sequence of the L19 antibody is shown in SEQ ID NO: 12. The sequence of L19 (ScFv) is shown in SEQ ID NO: 13. The sequence of L19 (Db) is shown in SEQ ID NO: 14.

An antigen binding site may comprise one, two, three, four, five or six CDRs of antibody L19. Amino acid sequences of the CDRs of L19 are:
SEQ ID NO:15 (CDR1 VH);
SEQ ID NO:16 (CDR2 VH);
SEQ ID NO:17 (CDR3 VH);
SEQ ID NO:18 (CDR1 VL);
SEQ ID NO:19 (CDR2 VL), and/or
SEQ ID NO:20 (CDR3 VL).

F16 is a human monoclonal antibody specific to the A1 domain of Tenascin C and has been previously described (WO2006/050834). The VH sequence of the F16 antibody is shown in SEQ ID NO: 21. The VL sequence of the F16 antibody is shown in SEQ ID NO: 22. The sequence of F16 (ScFv) is shown in SEQ ID NO: 23. The sequence of F16 (Db) is shown in SEQ ID NO: 24.

An antigen binding site may comprise one, two, three, four, five or six CDRs of antibody F16. Amino acid sequences of the CDRs of F16 are:
SEQ ID NO:25 (CDR1 VH);
SEQ ID NO:26 (CDR2 VH);
SEQ ID NO:27 (CDR3 VH);
SEQ ID NO:28 (CDR1 VL);
SEQ ID NO:29 (CDR2 VL), and/or
SEQ ID NO:30 (CDR3 VL).

A specific binding member may comprise a VH domain having at least 70%, more preferably one of at least 75%, 80%, 85%, 90%, 95%, 96%, 97%, 98%, 99% or 100%, sequence identity to the F8 VH domain amino acid sequence of SEQ ID NO: 1, the L19 VH domain amino acid sequence of SEQ ID NO: 11, or the F16 VH domain amino acid sequence of SEQ ID NO: 21.

A specific binding member may comprise have a VL domain having at least 70%, more preferably one of at least 75%, 80%, 85%, 90%, 95%, 96%, 97%, 98%, 99% or 100%, sequence identity to the F8 VL domain amino acid sequence of SEQ ID NO: 2, the L19 VL domain amino acid sequence of SEQ ID NO: 12, or the F16 VL domain amino acid sequence of SEQ ID NO: 22.

Sequence identity is commonly defined with reference to the algorithm GAP (Wisconsin GCG package, Accelerys Inc, San Diego USA). GAP uses the Needleman and Wunsch algorithm to align two complete sequences that maximizes the number of matches and minimizes the number of gaps. Generally, default parameters are used, with a gap creation penalty=12 and gap extension penalty=4. Use of GAP may be preferred but other algorithms may be used, e.g. BLAST (which uses the method of Altschul et al. (1990) J. Mol. Biol. 215: 405-410), FASTA (which uses the method of Pearson and Lipman (1988) PNAS USA 85: 2444-2448), or the Smith-Waterman algorithm (Smith and Waterman (1981) J. Mol Biol. 147: 195-197), or the TBLASTN program, of Altschul et al. (1990) supra, generally employing default parameters. In particular, the psi-Blast algorithm (Altschul et al., Nucl. Acids Res. (1997) 25: 3389-3402) may be used.

Variants of these VH and VL domains and CDRs may also be employed in specific binding members for use in the conjugates described herein. Suitable variants can be obtained by means of methods of sequence alteration, or mutation, and screening. Particular variants for use as described herein may include one or more amino acid sequence alterations (addition, deletion, substitution and/or insertion of an amino acid residue), maybe less than about 20 alterations, less than about 15 alterations, less than about 10 alterations or less than about 5 alterations, 4, 3, 2 or 1. Alterations may be made in one or more framework regions and/or one or more CDRs. In particular, alterations may be made in VH CDR1, VH CDR2 and/or VH CDR3.

A ScFv for use in the invention may have at least 70%, more preferably one of at least 75%, 80%, 85%, 90%, 95%, 96%, 97%, 98%, 99% or 100%, sequence identity to the amino acid sequence of the F8 ScFv set forth in SEQ ID NO: 3 or to the amino acid sequence of the L19 ScFv set forth in SEQ ID NO: 13 or to the amino acid sequence of the F16 ScFv set forth in SEQ ID NO: 23.

A Db for use in the invention may have at least 70%, more preferably one of at least 75%, 80%, 85%, 90%, 95%, 96%, 97%, 98%, 99% or 100%, sequence identity to the amino acid sequence of the F8 Db set forth in SEQ ID NO: 4 or to the amino acid sequence of the L19 Db set forth in SEQ ID NO: 14 or to the amino acid sequence of the F16 Db set forth in SEQ ID NO: 24.

Preferably, the specific binding members comprise the CDRs, VH and/or VL domains, or the sequence of the F8 antibody.

The conjugate of the present invention comprises interleukin-4 (IL4). IL4 may be mammalian IL4, preferably human IL4. IL4 may comprise or consist of the sequence shown in SEQ ID NO: 31, SEQ ID NO: 32, or SEQ ID NO: 46. Typically, IL4 has at least 70%, more preferably one of at least 75%, 80%, 85%, 90%, 95%, 96%, 97%, 98%, 99% or 100%, sequence identity to the amino acid sequence shown in SEQ ID NO: 31, SEQ ID NO: 32 or SEQ ID NO: 46.

In WO2014/173570 the current applicants have shown that substitution of the asparagine residue at position 38 of SEQ ID NO: 31 with glutamine prevents glycosylation of IL4 at this residue. Substitution of the asparagine residue at position 38 of SEQ ID NO: 31 with serine or alanine is expected to similarly prevent glycosylation of IL4. It is generally preferable to avoid glycosylation, as glycosylation may interfere with conjugate production, including batch consistency, and result in more rapid clearance of the conjugate from the patient's body. Preferably, a conjugate of the present invention, and in particular the IL4 present in a conjugate of the present invention, is not glycosylated. Thus, IL4 may comprise or consist of the sequence shown in SEQ ID NO: 31, except that the residue at position 38 of SEQ ID NO: 31 is a serine, glutamine, or alanine residue rather than an asparagine residue. Preferably, IL4 comprises or consists of the sequence shown in SEQ ID NO: 31, except that the residue at position 38 of SEQ ID NO: 31 is a glutamine residue rather than an asparagine residue. This sequence is shown in SEQ ID NO: 32. Alternatively, IL4 may comprise or consist of the sequence shown in SEQ ID NO: 31, except that the residue at position 38 of SEQ ID NO: 31 is a serine residue rather than an asparagine residue. As a further alternative, IL4 may comprise or consist of the sequence shown in SEQ ID NO: 31, except that the residue at position 38 of SEQ ID NO: 31 is an alanine residue rather than an asparagine residue. Occasionally IL4 may also be glycosylated at position 105 of SEQ ID NO: 31. Thus, in addition to the mutations mentioned above, the residue at position 105 of SEQ ID NO: 31 may be a serine, glutamine, or alanine residue rather than an asparagine residue, in order to prevent glycosylation at this position.

IL4 in conjugates of the invention retains a biological activity of IL4, e.g. anti-inflammatory activity; the ability to inhibit cell proliferation and/or differentiation; the ability to induce apoptosis; the ability to stimulate the proliferation of activated B cells and T cells; the ability to induce the differentiation of naïve helper T cells into Th2 cells after antigen challenge; the ability to stimulate the proliferation of NK cells; the ability to up-regulate MHC class II production; and/or the ability to inhibit tumour growth and/or metastasis.

The peptide linker linking the specific binding members and IL4 may be a flexible peptide linker. Suitable examples of peptide linker sequences are known in the art. The linker may be 10-20 amino acids, preferably 15-20 amino acids in length. Most preferably, the linker is 15 amino acids in length. Most preferably, the linker has the sequence SSSSGSSSSGSSSSG (SEQ ID NO: 33).

In a preferred embodiment, the conjugate of the present invention may comprise or consist of the sequence shown in SEQ ID NO: 35 (ScFvF8-[human] IL4-ScFvF8). The conjugate may have at least 70%, more preferably one of at least 75%, 80%, 85%, 90%, 95%, 96%, 97%, 98%, 99% or 100%, sequence identity to the amino acid sequence shown in SEQ ID NO: 35.

In another preferred embodiment, the conjugate of the present invention may comprise or consist of the sequence shown in SEQ ID NO: 36 (ScFvF8-[human] IL4 N38Q-ScFvF8), in which human IL4 is not glycosylated. The conjugate may have at least 70%, more preferably one of at least 75%, 80%, 85%, 90%, 95%, 96%, 97%, 98%, 99% or 100%, sequence identity to the amino acid sequence shown in SEQ ID NO: 36, provided the IL4 is not glycosylated.

The present inventors have shown that the conjugates of the present invention can be successfully used for the in vivo targeting of certain conditions and diseases, in particular conditions and diseases which are characterised by expression of the ED-A isoform of fibronectin, the ED-B isoform of fibronectin, and/or alternatively spliced tenascin C. Expression in this context may refer to over-expression compared with expression of the protein in normal tissue.

The ED-A isoform of fibronectin, the ED-B isoform of fibronectin, and alternatively spliced tenascin C are associated with neoplastic growth and/or angiogenesis. Accordingly, a conjugate according to the present invention may be used in a method of inhibiting angiogenesis in a patient by targeting IL4 to the neovasculature in vivo. A conjugate according to the present invention may also be used in a method of delivering IL4 to sites of neovasculature, which are the result of angiogenesis and/or tissue remodelling, in a patient. A method of inhibiting angiogenesis by targeting IL4 to sites of neovasculature in a patient, the method comprising administering a therapeutically effective amount of a conjugate according to the present invention, and a method of delivering a IL4 to sites of neovasculature, which are the result of angiogenesis, in a human or animal comprising administering to the human or animal a specific binding member according to the present invention, are also provided. Further provided is the use of a conjugate of the present invention for the preparation, or manufacture of a medicament for inhibiting angiogenesis, as well as the use of a conjugate of the present invention for the preparation, or manufacture, of a medicament for delivering IL4 to sites of neovasculature which are the result of angiogenesis, in a patient.

A conjugate may comprise two specific binding members and an interleukin, such as IL4. The specific binding members are preferably antibody molecules, most preferably single chain Fvs, as described herein. Where the conjugate comprises single chain Fvs, one or both of the single chain Fvs (scFvs) may be conjugated to the interleukin, e.g. IL4. An scFv may be conjugated to the interleukin, such as IL4, by means of a peptide linker, allowing the scFv-interleukin construct to be expressed as a fusion protein. By “fusion protein” is meant a polypeptide that is a translation product resulting from the fusion of two or more genes or nucleic acid coding sequences into one open reading frame (ORF). The fused expression products of the two genes in the ORF may be conjugated by a peptide linker encoded in-frame. Suitable peptide linkers are described herein.

“Specific binding member” describes one member of a pair of molecules that bind specifically to one another. The members of a specific binding pair may be naturally derived or wholly or partially synthetically produced. One member of the pair of molecules has an area on its surface, or a cavity, which binds to and is therefore complementary to a particular spatial and polar organization of the other member of the pair of molecules. Examples of types of binding pairs are antigen-antibody, biotin-avidin, hormone-hormone receptor, receptor-ligand, enzyme-substrate. The present invention is concerned with antigen-antibody type reactions.

A specific binding member normally comprises a molecule having an antigen-binding site. For example, a specific binding member may be an antibody molecule or a non-antibody protein that comprises an antigen-binding site. A specific binding member, as referred to herein, is preferably an antibody molecule.

An antigen binding site may be provided by means of arrangement of complementarity determining regions (CDRs) on non-antibody protein scaffolds such as fibronectin or cytochrome B etc. (Haan & Maggos, (2004), BioCentury, 12(5): A1-A6; Koide et al., (1998), Journal of Molecular Biology, 284: 1141-1151; Nygren et al., (1997), Current Opinion in Structural Biology, 7: 463-469), or by randomising or mutating amino acid residues of a loop within a protein scaffold to confer binding specificity for a desired target. Scaffolds for engineering novel binding sites in proteins have been reviewed in detail by Nygren et al. (1997) (Current Opinion in Structural Biology, 7: 463-469). Protein scaffolds for antibody mimics are disclosed in WO/0034784, in which the inventors describe proteins (antibody mimics) that include a fibronectin type III domain having at least one randomised loop. A suitable scaffold into which to graft one or more CDRs, e.g. a set of HCDRs, may be provided by any domain member of the immunoglobulin gene superfamily. The scaffold may be a human or non-human protein. An advantage of a non-antibody protein scaffold is that it may provide an antigen-binding site in a scaffold molecule that is smaller and/or easier to manufacture than at least some antibody molecules. Small size of a specific binding member may confer useful physiological properties such as an ability to enter cells, penetrate deep into tissues or reach targets within other structures, or to bind within protein cavities of the target antigen. Use of antigen binding sites in non-antibody protein scaffolds is reviewed in Wess, 2004, in BioCentury, The Bernstein Report on BioBusiness, 12(42), A1-A7. Typical are proteins having a stable backbone and one or more variable loops, in which the amino acid sequence of the loop or loops is specifically or randomly mutated to create an antigen-binding site that binds the target antigen. Such proteins include the IgG-binding domains of protein A from S. aureus, transferrin, tetranectin, fibronectin (e.g. 10th fibronectin type III domain) and lipocalins. Other approaches include synthetic “Microbodies” (Selecore GmbH), which are based on cyclotides—small proteins having intra-molecular disulphide bonds.

In addition to antibody sequences and/or an antigen-binding site, a specific binding member for use in the present invention may comprise other amino acids, e.g. forming a peptide or polypeptide, such as a folded domain, or to impart to the molecule another functional characteristic in addition to ability to bind antigen.

For example, a specific binding member may comprise a catalytic site (e.g. in an enzyme domain) as well as an antigen binding site, wherein the antigen binding site binds to the antigen and thus targets the catalytic site to the antigen. The catalytic site may inhibit biological function of the antigen, e.g. by cleavage.

Although, as noted, CDRs can be carried by non-antibody scaffolds, the structure for carrying a CDR or a set of CDRs will generally be an antibody heavy or light chain sequence or substantial portion thereof in which the CDR or set of CDRs is located at a location corresponding to the CDR or set of CDRs of naturally occurring VH and VL antibody variable domains encoded by rearranged immunoglobulin genes.

A number of different systems are used in the art to define the CDR residues within the antibody variable domains. The most commonly used systems are Kabat, Chothia, AbM, and Contact Definition. The Kabat CDR definition (Kabat et al., “Sequences of Proteins of Immunological Interest,” National Institutes of Health, Bethesda, Md. (1991)) is based upon antibody sequence variability. The Chothia CDR definition (Chothia et al., “Canonical structures for the hypervariable regions of immunoglobulins”, Journal of Molecular Biology, 196, 901-917 (1987); Al-Lazikani et al., “Standard conformations for the canonical structures of immunoglobulins”, Journal of Molecular Biology, 273, 927-948 (1997)) is based on three-dimensional structures of antibodies and topologies of the CDR loops. The AbM is a compromise between Kabat and Chothia using the Oxford Molecular's AbM modeling software. The Contact Definition is based on the analysis of the crystal structures. Any of these systems may be used to define individual CDRs.

The term CDR or CDRs is used here in order to indicate, according to the case, one of these regions or several, or even the whole, of these regions which contain the majority of the amino acid residues responsible for the binding by affinity of the antibody for the antigen or the epitope which it recognizes.

Among the six short CDR sequences, the third CDR of the heavy chain (HCDR3) has a greater size variability (greater diversity essentially due to the mechanisms of arrangement of the genes which give rise to it). It can be as short as 2 amino acids although the longest size known is 26. Functionally, HCDR3 plays a role in part in the determination of the specificity of the antibody (Segal et al., (1974), PNAS, 71:4298-4302; Amit et al., (1986), Science, 233:747-753; Chothia et al., (1987), J. Mol. Biol., 196:901-917; Chothia et al., (1989), Nature, 342:877-883; Caton et al., (1990), J. Immunol., 144:1965-1968; Sharon et al., (1990a), PNAS, 87:4814-4817; Sharon et al., (1990b), J. Immunol., 144:4863-4869; Kabat et al., (1991b), J. Immunol., 147:1709-1719).

The term “antibody molecule” describes an immunoglobulin whether natural or partly or wholly synthetically produced. The term also relates to any polypeptide or protein comprising an antibody antigen-binding site. It must be understood here that the invention does not relate to the antibodies in natural form, that is to say they are not in their natural environment but that they have been able to be isolated or obtained by purification from natural sources, or else obtained by genetic recombination, or by chemical synthesis, and that they can then contain unnatural amino acids as will be described later. Antibody fragments that comprise an antibody antigen-binding site include, but are not limited to, antibody molecules such as Fab, Fab′, Fab′-SH, scFv, Fv, dAb, Fd; and diabodies. A specific binding member, or antibody, for use in the present invention preferably comprises an scFv or is a diabody, a single chain diabody. Most preferably, a specific binding member, or antibody, for use in the present invention is an scFv.

It is possible to take monoclonal and other antibodies and use techniques of recombinant DNA technology to produce other antibodies or chimeric molecules that bind the target antigen. Such techniques may involve introducing DNA encoding the immunoglobulin variable region, or the CDRs, of an antibody to the constant regions, or constant regions plus framework regions, of a different immunoglobulin. See, for instance, EP-A-184187, GB 2188638A or EP-A-239400, and a large body of subsequent literature. A hybridoma or other cell producing an antibody may be subject to genetic mutation or other changes, which may or may not alter the binding specificity of antibodies produced.

As antibodies can be modified in a number of ways, the term “antibody molecule” should be construed as covering any specific binding member or substance having an antibody antigen-binding site with the required specificity and/or binding to antigen. Thus, this term covers antibody fragments and derivatives, including any polypeptide comprising an antibody antigen-binding site, whether natural or wholly or partially synthetic. Chimeric molecules comprising an antibody antigen-binding site, or equivalent, fused to another polypeptide (e.g. derived from another species or belonging to another antibody class or subclass) are therefore included. Cloning and expression of chimeric antibodies are described in EP-A-0120694 and EP-A-0125023, and a large body of subsequent literature.

Further techniques available in the art of antibody engineering have made it possible to isolate human and humanised antibodies. For example, human hybridomas can be made as described by Kontermann & Dubel (2001), S, Antibody Engineering, Springer-Verlag New York, LLC; ISBN: 3540413545. Phage display, another established technique for generating specific binding members has been described in detail in many publications such as WO92/01047 (discussed further below) and U.S. Pat. Nos. 5,969,108, 5,565,332, 5,733,743, 5,858,657, 5,871,907, 5,872,215, 5,885,793, 5,962,255, 6,140,471, 6,172,197, 6,225,447, 6,291,650, 6,492,160, 6,521,404 and Kontermann & Dubel (2001), S, Antibody Engineering, Springer-Verlag New York, LLC; ISBN: 3540413545. Transgenic mice in which the mouse antibody genes are inactivated and functionally replaced with human antibody genes while leaving intact other components of the mouse immune system, can be used for isolating human antibodies (Mendez et al., (1997), Nature Genet, 15(2): 146-156).

Synthetic antibody molecules may be created by expression from genes generated by means of oligonucleotides synthesized and assembled within suitable expression vectors, for example as described by Knappik et al. (2000) J. Mol. Biol. 296, 57-86 or Krebs et al. (2001) Journal of Immunological Methods, 254 67-84.

It has been shown that fragments of a whole antibody can perform the function of binding antigens. Examples of binding fragments are (i) the Fab fragment consisting of VL, VH, CL and CH1 domains; (ii) the Fd fragment consisting of the VH and CH1 domains; (iii) the Fv fragment consisting of the VL and VH domains of a single antibody; (iv) the dAb fragment (Ward et al. (1989) Nature 341, 544-546; McCafferty et al., (1990) Nature, 348, 552-554; Holt et al. (2003) Trends in Biotechnology 21, 484-490), which consists of a VH or a VL domain; (v) isolated CDR regions; (vi) F(ab′)2 fragments, a bivalent fragment comprising two linked Fab fragments (vii) single chain Fv molecules (scFv), wherein a VH domain and a VL domain are linked by a peptide linker which allows the two domains to associate to form an antigen binding site (Bird et al. (1988) Science, 242, 423-426; Huston et al. (1988) PNAS USA, 85, 5879-5883); (viii) bispecific single chain Fv dimers (PCT/US92/09965) and (ix) “diabodies”, multivalent or multispecific fragments constructed by gene fusion (WO94/13804; Holliger et al. (1993a), Proc. Natl. Acad. Sci. USA 90 6444-6448). Fv, scFv or diabody molecules may be stabilized by the incorporation of disulphide bridges linking the VH and VL domains (Reiter et al. (1996), Nature Biotech, 14, 1239-1245). Minibodies comprising a scFv joined to a CH3 domain may also be made (Hu et al. (1996), Cancer Res., 56(13):3055-61). Other examples of binding fragments are Fab′, which differs from Fab fragments by the addition of a few residues at the carboxyl terminus of the heavy chain CH1 domain, including one or more cysteines from the antibody hinge region, and Fab′-SH, which is a Fab′ fragment in which the cysteine residue(s) of the constant domains bear a free thiol group.

Antibody fragments for use in the invention can be obtained starting from any of the antibody molecules described herein, e.g. antibody molecules comprising VH and/or VL domains or CDRs of any of antibodies described herein, by methods such as digestion by enzymes, such as pepsin or papain and/or by cleavage of the disulfide bridges by chemical reduction.

In another manner, antibody fragments of the present invention may be obtained by techniques of genetic recombination likewise well known to the person skilled in the art or else by peptide synthesis by means of, for example, automatic peptide synthesizers such as those supplied by the company Applied Biosystems, etc., or by nucleic acid synthesis and expression.

Functional antibody fragments according to the present invention include any functional fragment whose half-life is increased by a chemical modification, especially by PEGylation, or by incorporation in a liposome.

A dAb (domain antibody) is a small monomeric antigen-binding fragment of an antibody, namely the variable region of an antibody heavy or light chain (Holt et al. (2003) Trends in Biotechnology 21, 484-490). VH dAbs occur naturally in camelids (e.g. camel, llama) and may be produced by immunizing a camelid with a target antigen, isolating antigen-specific B cells and directly cloning dAb genes from individual B cells. dAbs are also producible in cell culture. Their small size, good solubility and temperature stability makes them particularly physiologically useful and suitable for selection and affinity maturation. A specific binding member of the present invention may be a dAb comprising a VH or VL domain substantially as set out herein, or a VH or VL domain comprising a set of CDRs substantially as set out herein.

As used herein, the phrase “substantially as set out” refers to the characteristic(s) of the relevant CDRs of the VH or VL domain of specific binding members described herein will be either identical or highly similar to the specified regions of which the sequence is set out herein. As described herein, the phrase “highly similar” with respect to specified region(s) of one or more variable domains, it is contemplated that from 1 to about 5, e.g. from 1 to 4, including 1 to 3, or 1 or 2, or 3 or 4, amino acid substitutions may be made in the CDR and/or VH or VL domain.

Bispecific or bifunctional antibodies form a second generation of monoclonal antibodies in which two different variable regions are combined in the same molecule (Holliger and Bohlen 1999 Cancer and metastasis rev. 18: 411-419). Their use has been demonstrated both in the diagnostic field and in the therapy field from their capacity to recruit new effector functions or to target several molecules on the surface of tumor cells. Where bispecific antibodies are to be used, these may be conventional bispecific antibodies, which can be manufactured in a variety of ways (Holliger et al. (1993b), Current Opinion Biotechnol 4, 446-449), e.g. prepared chemically or from hybrid hybridomas, or may be any of the bispecific antibody fragments mentioned above. These antibodies can be obtained by chemical methods (Glennie et al., (1987) J. Immunol. 139, 2367-2375; Repp et al., (1995) J. Hemat. 377-382) or somatic methods (Staerz U. D. and Bevan M. J. (1986) PNAS 83; Suresh et al. (1986) Method. Enzymol. 121: 210-228) but likewise by genetic engineering techniques which allow the heterodimerization to be forced and thus facilitate the process of purification of the antibody sought (Merchand et al., 1998 Nature Biotech. 16: 677-681). Examples of bispecific antibodies include those of the BiTE™ technology in which the binding domains of two antibodies with different specificity can be used and directly linked via short flexible peptides. This combines two antibodies on a short single polypeptide chain. Diabodies and scFv can be constructed without an Fc region, using only variable domains, potentially reducing the effects of anti-idiotypic reaction.

Bispecific antibodies can be constructed as entire IgG, as bispecific Fab′2, as Fab′PEG, as diabodies or else as bispecific scFv. Further, two bispecific antibodies can be linked using routine methods known in the art to form tetravalent antibodies.

Bispecific diabodies, as opposed to bispecific whole antibodies, may also be particularly useful because they can be readily constructed and expressed in E. coli. Diabodies (and many other polypeptides such as antibody fragments) of appropriate binding specificities can be readily selected using phage display (WO94/13804) from libraries. If one arm of the diabody is to be kept constant, for instance, with a specificity directed against a target antigen, then a library can be made where the other arm is varied and an antibody of appropriate specificity selected. Bispecific whole antibodies may be made by alternative engineering methods as described in Ridgeway et al. (1996), Protein Eng., 9, 616-621.

Various methods are available in the art for obtaining antibodies against a target antigen. The antibodies may be monoclonal antibodies, especially of human, murine, chimeric or humanized origin, which can be obtained according to the standard methods well known to the person skilled in the art.

In general, for the preparation of monoclonal antibodies or their functional fragments, especially of murine origin, it is possible to refer to techniques which are described in particular in the manual “Antibodies” (Harlow and Lane, Antibodies: A Laboratory Manual, Cold Spring Harbor Laboratory, Cold Spring Harbor N.Y., pp. 726, 1988) or to the technique of preparation from hybridomas described by Kohler and Milstein, 1975, Nature, 256:495-497.

Monoclonal antibodies can be obtained, for example, from an animal cell immunized against A-FN, B-FN, or tenascin C or a fragment thereof containing the epitope recognized by said monoclonal antibodies, e.g. a fragment comprising or consisting of ED-A, ED-B, the A1 Domain of Tenascin C, or a peptide fragment thereof. The A-FN, B-FN, or tenascin C, or a fragment thereof, can especially be produced according to the usual working methods, by genetic recombination starting with a nucleic acid sequence contained in the cDNA sequence coding for A-FN, B-FN, or tenascin C, or fragment thereof, or by peptide synthesis starting from a sequence of amino acids comprised in the peptide sequence of the B-FN, or tenascin C, and/or a fragment thereof.

Monoclonal antibodies can, for example, be purified on an affinity column on which A-FN, B-FN, or tenascin C, or a fragment thereof containing the epitope recognized by said monoclonal antibodies, e.g. a fragment comprising or consisting of ED-A, B-FN, or tenascin C, or a peptide fragment of ED-A, B-FN, or tenascin C has previously been immobilized. Monoclonal antibodies can be purified by chromatography on protein A and/or G, followed or not followed by ion-exchange chromatography aimed at eliminating the residual protein contaminants as well as the DNA and the LPS, in itself, followed or not followed by exclusion chromatography on sepharose gel in order to eliminate the potential aggregates due to the presence of dimers or of other multimers. The whole of these techniques may be used simultaneously or successively.

The term “antigen binding site” describes the part of a molecule that binds to and is complementary to all or part of the target antigen. In an antibody molecule, it is referred to as the antibody antigen-binding site, and comprises the part of the antibody that binds to and is complementary to all or part of the target antigen. Where an antigen is large, an antibody may only bind to a particular part of the antigen, which part is termed an epitope. An antibody antigen-binding site may be provided by one or more antibody variable domains. An antibody antigen-binding site may comprise an antibody light chain variable region (VL) and an antibody heavy chain variable region (VH).

The term “isolated” refers to the state in which specific binding members for use in the invention or nucleic acid encoding such specific binding members, will generally be in accordance with the present invention. Thus, specific binding members, VH and/or VL domains of the present invention may be provided isolated and/or purified, e.g. from their natural environment, in substantially pure or homogeneous form, or, in the case of nucleic acid, free or substantially free of nucleic acid or genes of origin other than the sequence encoding a polypeptide with the required function. Isolated members and isolated nucleic acid will be free or substantially free of material with which they are naturally associated such as other polypeptides or nucleic acids with which they are found in their natural environment, or the environment in which they are prepared (e.g. cell culture) when such preparation is by recombinant DNA technology practised in vitro or in vivo. Specific binding members and nucleic acid may be formulated with diluents or adjuvants and still for practical purposes be isolated—for example the members will normally be mixed with gelatin or other carriers if used to coat microtitre plates for use in immunoassays, or will be mixed with pharmaceutically acceptable carriers or diluents when used in diagnosis or therapy. Specific binding members may be glycosylated, either naturally or by systems of heterologous eukaryotic cells (e.g. CHO or NS0 (ECACC 85110503) cells, or they may be (for example if produced by expression in a prokaryotic cell) unglycosylated.

Heterogeneous preparations comprising antibody molecules may also be used in the invention. For example, such preparations may be mixtures of antibodies with full-length heavy chains and heavy chains lacking the C-terminal lysine, with various degrees of glycosylation and/or with derivatized amino acids, such as cyclization of an N-terminal glutamic acid to form a pyroglutamic acid residue.

One or more specific binding members for an antigen, e.g. the A-FN, the ED-A, B-FN, the ED-B, tenascin C, or the A1 domain of tenascin C may be obtained by bringing into contact a library of specific binding members and the antigen or a fragment thereof, e.g. a fragment comprising or consisting of ED-A, ED-B, or the A1 domain of tenascin C, or a peptide fragment thereof, and selecting one or more specific binding members of the library able to bind the antigen.

An antibody library may be screened using Iterative Colony Filter Screening (ICFS as described in e.g. WO0246455.

A library may also be displayed on particles or molecular complexes, e.g. replicable genetic packages such bacteriophage (e.g. T7) particles, or other in vitro display systems, each particle or molecular complex containing nucleic acid encoding the antibody VH variable domain displayed on it, and optionally also a displayed VL domain if present. Phage display is described in WO92/01047 and e.g. US patents U.S. Pat. Nos. 5,969,108, 5,565,332, 5,733,743, 5,858,657, 5,871,907, 5,872,215, 5,885,793, 5,962,255, 6,140,471, 6,172,197, 6,225,447, 6,291,650, 6,492,160 and 6,521,404.

Following selection of specific binding members able to bind the antigen and displayed on bacteriophage or other library particles or molecular complexes, nucleic acid may be taken from a bacteriophage or other particle or molecular complex displaying a said selected specific binding member. Such nucleic acid may be used in subsequent production of a specific binding member or an antibody VH or VL variable domain by expression from nucleic acid with the sequence of nucleic acid taken from a bacteriophage or other particle or molecular complex displaying a said selected specific binding member.

Ability to bind an extra-cellular matrix component associated with neoplastic growth and/or angiogenesis, such as the A-FN, B-FN, the ED-A, or the ED-B of fibronectin, tenascin C or the A1 domain of tenascin C or other target antigen or isoform may be further tested, e.g. ability to compete with an antibody specific for the A-FN, B-FN, the ED-A, or the ED-B of fibronectin, tenascin C or the A1 domain of tenascin C, such as antibody F8, L19, or F16.

A specific binding member for use in the invention may bind an extra-cellular matrix component associated with neoplastic growth and/or angiogenesis, such as the A-FN, B-FN, the ED-A, or the ED-B of fibronectin, tenascin C or the A1 domain of tenascin C specifically. A specific binding member of the present invention may bind the A-FN and/or the ED-A of fibronectin, with the same affinity as anti-ED-A antibody F8 e.g. in scFv format, or with an affinity that is higher. A specific binding member of the present invention may bind the B-FN and/or the ED-B of fibronectin, with the same affinity as anti-ED-B antibody L19 e.g. in scFv format, or with an affinity that is higher. A specific binding member of the present invention may bind the Tenascin C and/or the A1 domain of tenascin C, with the same affinity as anti-ED-A antibody F16 e.g. in scFv format, or with an affinity that is higher.

A specific binding member of the present invention may bind to the same epitope on A-FN and/or the ED-A of fibronectin as anti-ED-A antibody F8. A specific binding member of the present invention may bind to the same epitope on B-FN and/or the ED-B of fibronectin as anti-ED-A antibody L19. A specific binding member of the present invention may bind to the same epitope on tenascin C and/or the A1 domain of tenascin C as antibody F16.

Variants of antibody molecules disclosed herein may be produced and used in the present invention. The techniques required to make substitutions within amino acid sequences of CDRs, antibody VH or VL domains, in particular the framework regions of the VH and VL domains, and specific binding members generally are available in the art. Variant sequences may be made, with substitutions that may or may not be predicted to have a minimal or beneficial effect on activity, and tested for ability to bind A-FN and/or the ED-A of fibronectin, B-FN and/or the ED-B of fibronectin, tenascin C and/or the A1 domain of tenascin C, and/or for any other desired property.

Variable domain amino acid sequence variants of any of the VH and VL domains whose sequences are specifically disclosed herein may be employed in accordance with the present invention, as discussed. Particular variants may include one or more amino acid sequence alterations (addition, deletion, substitution and/or insertion of an amino acid residue), may be less than about 20 alterations, less than about 15 alterations, less than about 10 alterations or less than about 5 alterations, maybe 5, 4, 3, 2 or 1. Alterations may be made in one or more framework regions and/or one or more CDRs. The alterations normally do not result in loss of function, so a specific binding member comprising a thus-altered amino acid sequence may retain an ability to bind A-FN and/or the ED-A of fibronectin, B-FN and/or the ED-B of fibronectin, tenascin C and/or the A1 domain of tenascin C. For example, it may retain the same quantitative binding as a specific binding member in which the alteration is not made, e.g. as measured in an assay described herein. The specific binding member comprising a thus-altered amino acid sequence may have an improved ability to bind A-FN and/or the ED-A of fibronectin, B-FN and/or the ED-B of fibronectin, tenascin C and/or the A1 domain of tenascin C. For example, a specific binding member that binds the ED-A isoform or ED-A of fibronectin, as referred to herein, may comprise the VH domain shown in SEQ ID NO: 1 and/or the VL domain shown in SEQ ID NO: 2 with 10 or fewer, for example, 5, 4, 3, 2 or 1 amino acid substitution within the framework region of the VH and/or VL domain. Such a specific binding member may bind the ED-A isoform or ED-A of fibronectin with the same or substantially the same, affinity as a specific binding member comprising the VH domain shown in SEQ ID NO: 1 and the VL domain shown in SEQ ID NO: 2 or may bind the ED-A isoform or ED-A of fibronectin with a higher affinity than a specific binding member comprising the VH domain shown in SEQ ID NO: 1 and the VL domain shown in SEQ ID NO: 2. A specific binding member that binds the ED-B isoform or ED-B of fibronectin, as referred to herein, may comprise the VH domain shown in SEQ ID NO: 11 and/or the VL domain shown in SEQ ID NO: 12 with 10 or fewer, for example, 5, 4, 3, 2 or 1 amino acid substitution within the framework region of the VH and/or VL domain. Such a specific binding member may bind the ED-B isoform or ED-B of fibronectin with the same or substantially the same, affinity as a specific binding member comprising the VH domain shown in SEQ ID NO: 11 and the VL domain shown in SEQ ID NO: 12 or may bind the ED-B isoform or ED-B of fibronectin with a higher affinity than a specific binding member comprising the VH domain shown in SEQ ID NO: 11 and the VL domain shown in SEQ ID NO:12. A specific binding member that binds tenascin C or the A1 domain of tenascin C, as referred to herein, may comprise the VH domain shown in SEQ ID NO: 21 and/or the VL domain shown in SEQ ID NO: 22 with 10 or fewer, for example, 5, 4, 3, 2 or 1 amino acid substitution within the framework region of the VH and/or VL domain. Such a specific binding member may bind tenascin C or the A1 domain of tenascin C with the same or substantially the same, affinity as a specific binding member comprising the VH domain shown in SEQ ID NO: 21 and the VL domain shown in SEQ ID NO: 22 or may bind tenascin C or the A1 domain of tenascin C with a higher affinity than a specific binding member comprising the VH domain shown in SEQ ID NO: 21 and the VL domain shown in SEQ ID NO: 22.

Novel VH or VL regions carrying CDR-derived sequences for use in the invention may be generated using random mutagenesis of one or more selected VH and/or VL genes to generate mutations within the entire variable domain. In some embodiments one or two amino acid substitutions are made within an entire variable domain or set of CDRs. Another method that may be used is to direct mutagenesis to CDR regions of VH or VL genes.

As noted above, a CDR amino acid sequence substantially as set out herein may be carried as a CDR in a human antibody variable domain or a substantial portion thereof. The HCDR3 sequences substantially as set out herein represent embodiments of the present invention and for example each of these may be carried as a HCDR3 in a human heavy chain variable domain or a substantial portion thereof.

Variable domains employed in the invention may be obtained or derived from any germ-line or rearranged human variable domain, or may be a synthetic variable domain based on consensus or actual sequences of known human variable domains. A variable domain can be derived from a non-human antibody. A CDR sequence for use in the invention (e.g. CDR3) may be introduced into a repertoire of variable domains lacking a CDR (e.g. CDR3), using recombinant DNA technology. For example, Marks et al. (1992) describe methods of producing repertoires of antibody variable domains in which consensus primers directed at or adjacent to the 5′ end of the variable domain area are used in conjunction with consensus primers to the third framework region of human VH genes to provide a repertoire of VH variable domains lacking a CDR3. Marks et al. further describe how this repertoire may be combined with a CDR3 of a particular antibody. Using analogous techniques, the CDR3-derived sequences of the present invention may be shuffled with repertoires of VH or VL domains lacking a CDR3, and the shuffled complete VH or VL domains combined with a cognate VL or VH domain to provide specific binding members for use in the invention. The repertoire may then be displayed in a suitable host system such as the phage display system of WO92/01047, or any of a subsequent large body of literature, including Kay, Winter & McCafferty (1996), so that suitable specific binding members may be selected. A repertoire may consist of from anything from 10⁴ individual members upwards, for example at least 10⁵, at least 10⁶, at least 107, at least 10⁸, at least 10⁹ or at least 10¹⁰ members.

Similarly, one or more, or all three CDRs may be grafted into a repertoire of VH or VL domains that are then screened for a specific binding member or specific binding members for A-FN, B-FN, the ED-A, or the ED-B of fibronectin, tenascin C or the A1 domain of tenascin C.

An antibody molecule of, or for use, in the invention may comprise the HCDR1, HCDR2, HCDR3, LCDR1, LCDR2, and/or LCDR3 sequences of an antibody as disclosed or described herein in a framework. The frameworks are preferably human frameworks, specifically human germline frameworks. Thus a VH and/or VL domain framework, as referred to herein, is preferably a human framework, more preferably a human germline framework. For example, the VH domain framework may be DP47 and/or the VL domain framework may be DPL16 or DPK22.

One or more of the HCDR1, HCDR2 and HCDR3 of antibody F8, L19, or F16, or the set of HCDRs of antibody F8, L19, or F16 may be employed, and/or one or more of the LCDR1, LCDR2 and LCDR3 of antibody F8, L19, or F16, or the set of LCDRs of antibody F8, L19, or F16 may be employed.

Similarly, other VH and VL domains, sets of CDRs and sets of HCDRs and/or sets of LCDRs disclosed herein may be employed.

An extra-cellular matrix component associated with neoplastic growth and/or angiogenesis, such as the A-FN, B-FN, the ED-A, or the ED-B of fibronectin, tenascin C or the A1 domain of tenascin C may be used in a screen for specific binding members, e.g. antibody molecules, for use in the invention. The screen may a screen of a repertoire as disclosed elsewhere herein.

A substantial portion of an immunoglobulin variable domain may comprise at least the three CDR regions, together with their intervening framework regions. The portion may also include at least about 50% of either or both of the first and fourth framework regions, the 50% being the C-terminal 50% of the first framework region and the N-terminal 50% of the fourth framework region. Additional residues at the N-terminal or C-terminal end of the substantial part of the variable domain may be those not normally associated with naturally occurring variable domain regions. For example, construction of specific binding members of the present invention made by recombinant DNA techniques may result in the introduction of N- or C-terminal residues encoded by linkers introduced to facilitate cloning or other manipulation steps. Other manipulation steps include the introduction of linkers to join variable domains disclosed elsewhere herein to further protein sequences including antibody constant regions, other variable domains (for example in the production of diabodies) or detectable/functional labels as discussed in more detail elsewhere herein.

Although specific binding members may comprise a pair of VH and VL domains, single binding domains based on either VH or VL domain sequences may also be used in the invention. It is known that single immunoglobulin domains, especially VH domains, are capable of binding target antigens in a specific manner. For example, see the discussion of dAbs above.

In the case of either of the single binding domains, these domains may be used to screen for complementary domains capable of forming a two-domain specific binding member able to bind an extra-cellular matrix component associated with neoplastic growth and/or angiogenesis, such as, the ED-A, or the ED-B domain of fibronectin, or the A1 domain of tenascin C. This may be achieved by phage display screening methods using the so-called hierarchical dual combinatorial approach as disclosed in WO92/01047, in which an individual colony containing either an H or L chain clone is used to infect a complete library of clones encoding the other chain (L or H) and the resulting two-chain specific binding member is selected in accordance with phage display techniques such as those described in that reference. This technique is also disclosed in Marks et al. (1992).

Specific binding members for use in the present invention may further comprise antibody constant regions or parts thereof, e.g. human antibody constant regions or parts thereof. For example, a VL domain may be attached at its C-terminal end to antibody light chain constant domains including human C kappa or C lambda chains, e.g. C lambda. Similarly, a specific binding member based on a VH domain may be attached at its C-terminal end to all or part (e.g. a CH1 domain) of an immunoglobulin heavy chain derived from any antibody isotype, e.g. IgG, IgA, IgE and IgM and any of the isotype sub-classes, particularly IgG1 and IgG4. Any synthetic or other constant region variant that has these properties and stabilizes variable regions is also useful in embodiments of the present invention.

In the context of the present invention, a specific binding member (e.g. antibody), as described herein, forms part of a conjugate with IL4. The IL4 is preferably human IL4. The specific binding member preferably comprises an scFv.

The specific binding member and IL4 may be connected to each other directly, for example through any suitable chemical bond, or through a linker, for example a peptide linker. Where the specific binding member is linked to IL4 by means of a peptide linker, the conjugate may be fusion protein.

The chemical bond may be, for example, a covalent or ionic bond. Examples of covalent bonds include peptide bonds (amide bonds) and disulphide bonds. The specific binding member and IL4 may be covalently linked, for example by peptide bonds (amide bonds). Thus, the specific binding member, in particular an scFv portion of a specific binding member, and IL4 may be produces as a fusion protein. Where the specific binding member is a two-chain or multi-chain molecule, IL4 may be conjugated as a fusion polypeptide with one or more polypeptide chains in the specific binding member.

The peptide linker connecting the specific binding member and IL4 may be a flexible peptide linker. Suitable examples of peptide linker sequences are known in the art. The linker may be 10-20 amino acids, preferably 15-20 amino acids in length. Most preferably, the linker is 15 amino acids in length. Most preferably, the linker has the sequence SSSSGSSSSGSSSSG (SEQ ID NO: 33) or GGGGSGGGGSGGGGS (SEQ ID NO: 34).

Other means for conjugation include chemical conjugation, especially cross-linking using a bifunctional reagent (e.g. employing DOUBLE-REAGENTS™ Cross-linking Reagents Selection Guide, Pierce).

Also provided is an isolated nucleic acid molecule encoding a conjugate according to the present invention. Nucleic acid molecules may comprise DNA and/or RNA and may be partially or wholly synthetic. Reference to a nucleotide sequence as set out herein encompasses a DNA molecule with the specified sequence, and encompasses a RNA molecule with the specified sequence in which U is substituted for T, unless context requires otherwise.

Further provided are constructs in the form of plasmids, vectors, transcription or expression cassettes which comprise such nucleic acids. Suitable vectors can be chosen or constructed, containing appropriate regulatory sequences, including promoter sequences, terminator sequences, polyadenylation sequences, enhancer sequences, marker genes and other sequences as appropriate. Vectors may be plasmids e.g. phagemid, or viral e.g. ‘phage, as appropriate. For further details, see, for example, Sambrook & Russell (2001) Molecular Cloning: a Laboratory Manual: 3rd edition, Cold Spring Harbor Laboratory Press. Many known techniques and protocols for manipulation of nucleic acid, for example in the preparation of nucleic acid constructs, mutagenesis, sequencing, introduction of DNA into cells and gene expression, and analysis of proteins, are described in detail in Ausubel et al. (1999) 4^{th} eds., Short Protocols in Molecular Biology: A Compendium of Methods from Current Protocols in Molecular Biology, John Wiley & Sons.

A recombinant host cell that comprises one or more constructs as described above is also provided. Suitable host cells include bacteria, mammalian cells, plant cells, filamentous fungi, yeast and baculovirus systems and transgenic plants and animals.

A conjugate according to the present invention may be produced using such a recombinant host cell. The production method may comprise expressing a nucleic acid or construct as described above. Expression may conveniently be achieved by culturing the recombinant host cell under appropriate conditions for production of the conjugate. Following production, the conjugate may be isolated and/or purified using any suitable technique, and then used as appropriate. The conjugate may be formulated into a composition including at least one additional component, such as a pharmaceutically acceptable excipient.

Systems for cloning and expression of a polypeptide in a variety of different host cells are well known. The expression of antibodies, including conjugates thereof, in prokaryotic cells is well established in the art. For a review, see for example Plückthun (1991), Bio/Technology 9: 545-551. A common bacterial host is E. coli.

Expression in eukaryotic cells in culture is also available to those skilled in the art as an option for production of conjugates for example Chadd et al. (2001), Current Opinion in Biotechnology 12: 188-194); Andersen et al. (2002) Current Opinion in Biotechnology 13: 117; Larrick & Thomas (2001) Current Opinion in Biotechnology 12:411-418. Mammalian cell lines available in the art for expression of a heterologous polypeptide include Chinese hamster ovary (CHO) cells, HeLa cells, baby hamster kidney cells, NS0 mouse melanoma cells, YB2/0 rat myeloma cells, human embryonic kidney cells, human embryonic retina cells and many others.

A method comprising introducing a nucleic acid or construct disclosed herein into a host cell is also described. The introduction may employ any available technique. For eukaryotic cells, suitable techniques may include calcium phosphate transfection, DEAE-Dextran, electroporation, liposome-mediated transfection and transduction using retrovirus or other virus, e.g. vaccinia or, for insect cells, baculovirus. Introducing nucleic acid in the host cell, in particular a eukaryotic cell may use a viral or a plasmid based system. The plasmid system may be maintained episomally or may be incorporated into the host cell or into an artificial chromosome. Incorporation may be either by random or targeted integration of one or more copies at single or multiple loci. For bacterial cells, suitable techniques may include calcium chloride transformation, electroporation and transfection using bacteriophage.

The nucleic acid or construct may be integrated into the genome (e.g. chromosome) of the host cell. Integration may be promoted by inclusion of sequences that promote recombination with the genome, in accordance with standard techniques.

The conjugates of the present invention are designed to be used in methods of treatment of patients, preferably human patients. Conjugates of the present invention may be used in the treatment of a disease/disorder, such as cancer and/or autoimmune diseases, such as rheumatoid arthritis (RA), multiple sclerosis (MS), inflammatory bowel disease (IBD), psoriasis, psoriatic arthritis, endometriosis, Behçet's disease and periodontitis. Other diseases which may be treated or prevented using the conjugates of the invention include autoimmune insulitis and diabetes, in particular autoimmune diabetes. Polymorbid patients, i.e. patients suffering from more than one of these diseases may also be treated using the conjugates of the present invention.

Accordingly, the invention provides methods of treatment comprising administration of a conjugate according to the present invention, pharmaceutical compositions comprising such conjugate, and use of such a conjugate in the manufacture of a medicament for administration, for example in a method of making a medicament or pharmaceutical composition comprising formulating the conjugate with a pharmaceutically acceptable excipient. Pharmaceutically acceptable vehicles are well known and will be adapted by the person skilled in the art as a function of the nature and of the mode of administration of the active compound(s) chosen.

Conjugates according to the present invention will usually be administered in the form of a pharmaceutical composition, which may comprise at least one component in addition to the specific binding member. Thus, pharmaceutical compositions described herein, and for use in accordance with the present invention, may comprise, in addition to active ingredient, a pharmaceutically acceptable excipient, carrier, buffer, stabilizer or other materials well known to those skilled in the art. Such materials should be non-toxic and should not interfere with the efficacy of the active ingredient. The precise nature of the carrier or other material will depend on the route of administration, which may be by injection, e.g. intravenous or subcutaneous. Preferably, the conjugate of the present invention is administered intravenously, in particular where the disease to be treated or prevented is cancer, MS, IBD, psoriasis, psoriatic arthritis, periodontitis, endometriosis, Behçet's disease, insulitis or diabetes. Where the treatment concerns RA, the conjugate may be administered subcutaneously.

Liquid pharmaceutical compositions generally comprise a liquid carrier such as water, petroleum, animal or vegetable oils, mineral oil or synthetic oil. Physiological saline solution, dextrose or other saccharide solution or glycols such as ethylene glycol, propylene glycol or polyethylene glycol may be included.

For intravenous injection, or injection at the site of affliction, the active ingredient will be in the form of a parenterally acceptable aqueous solution which is pyrogen-free and has suitable pH, isotonicity and stability. Those of relevant skill in the art are well able to prepare suitable solutions using, for example, isotonic vehicles such as Sodium Chloride Injection, Ringer's Injection, Lactated Ringer's Injection. Preservatives, stabilizers, buffers, antioxidants and/or other additives may be employed, as required. Many methods for the preparation of pharmaceutical formulations are known to those skilled in the art. See e.g. Robinson ed., Sustained and Controlled Release Drug Delivery Systems, Marcel Dekker, Inc., New York, 1978.

A composition comprising a conjugate according to the present invention may be administered alone or in combination with other treatments, concurrently or sequentially or as a combined preparation with another therapeutic agent or agents, dependent upon the condition to be treated.

For example, a conjugate may be used in combination with an existing therapeutic agent for the disease to be treated.

In accordance with the present invention, compositions provided may be administered to mammals, preferably humans. Administration may be in a “therapeutically effective amount”, this being sufficient to show benefit to a patient. Such benefit may be at least amelioration of at least one symptom. Thus “treatment” of a specified disease refers to amelioration of at least one symptom. The actual amount administered, and rate and time-course of administration, will depend on the nature and severity of what is being treated, the particular patient being treated, the clinical condition of the individual patient, the cause of the disorder, the site of delivery of the composition, the type of conjugate, the method of administration, the scheduling of administration and other factors known to medical practitioners. Prescription of treatment, e.g. decisions on dosage etc., is within the responsibility of general practitioners and other medical doctors, and may depend on the severity of the symptoms and/or progression of a disease being treated. Appropriate doses of antibody are well known in the art (Ledermann et al. (1991) Int. J. Cancer 47: 659-664; and Bagshawe et al. (1991) Antibody, Immunoconjugates and Radiopharmaceuticals 4: 915-922). Specific dosages indicated herein, or in the Physician's Desk Reference (2003) as appropriate for the type of medicament being administered, may be used. A therapeutically effective amount or suitable dose of a conjugate for use in the invention can be determined by comparing its in vitro activity and in vivo activity in an animal model. Methods for extrapolation of effective dosages in mice and other test animals to humans are known. The precise dose will depend upon a number of factors, including whether the antibody is for diagnosis, prevention or for treatment, the size and location of the area to be treated, the precise nature of the conjugate. An initial higher loading dose, followed by one or more lower doses, may be administered. This is a dose for a single treatment of an adult patient, which may be proportionally adjusted for children and infants, and also adjusted according to conjugate format in proportion to molecular weight. Treatments may be repeated at daily, twice-weekly, weekly or monthly intervals, at the discretion of the physician. Treatments may be every two to four weeks for subcutaneous administration and every four to eight weeks for intravenous administration. In some embodiments of the present invention, treatment is periodic, and the period between administrations is about two weeks or more, e.g. about three weeks or more, about four weeks or more, or about once a month. In other embodiments of the invention, treatment may be given before, and/or after surgery, and may be administered or applied directly at the anatomical site of surgical treatment.

Fibronectin is an antigen subject to alternative splicing, and a number of alternative isoforms of fibronectin are known, including alternatively spliced isoforms A-FN and B-FN, comprising domains ED-A or ED-B respectively, which are known markers of angiogenesis. A specific binding member, as referred to herein, may selectively bind to isoforms of fibronectin selectively expressed in the neovasculature. A specific binding member may bind fibronectin isoform A-FN, e.g. it may bind domain ED-A (extra domain A). A specific binding member may bind ED-B (extra domain B).

Fibronectin Extra Domain-A (EDA or ED-A) is also known as ED, extra type III repeat A (EIIIA) or EDI. The sequence of human ED-A has been published by Kornblihtt et al. (1984), Nucleic Acids Res. 12, 5853-5868 and Paolella et al. (1988), Nucleic Acids Res. 16, 3545-3557. The sequence of human ED-A is also available on the SwissProt database as amino acids 1631-1720 (Fibronectin type-Ill 12; extra domain 2) of the amino acid sequence deposited under accession number P02751. The sequence of mouse ED-A is available on the SwissProt database as amino acids 1721-1810 (Fibronectin type-Ill 13; extra domain 2) of the amino acid sequence deposited under accession number P11276.

The ED-A isoform of fibronectin (A-FN) contains the Extra Domain-A (ED-A). The sequence of the human A-FN can be deduced from the corresponding human fibronectin precursor sequence which is available on the SwissProt database under accession number P02751.

The sequence of the mouse A-FN can be deduced from the corresponding mouse fibronectin precursor sequence which is available on the SwissProt database under accession number P11276. The A-FN may be the human ED-A isoform of fibronectin. The ED-A may be the Extra Domain-A of human fibronectin.

ED-A is a 90 amino acid sequence which is inserted into fibronectin (FN) by alternative splicing and is located between domain 11 and 12 of FN (Borsi et al. (1987), J. Cell. Biol., 104, 595-600). ED-A is mainly absent in the plasma form of FN but is abundant during embryogenesis, tissue remodelling, fibrosis, cardiac transplantation and solid tumour growth.

Fibronectin isoform B-FN is one of the best known markers angiogenesis (U.S. Ser. No. 10/382,107, WO01/62298). An extra domain “ED-B” of 91 amino acids is found in the B-FN isoform and is identical in mouse, rat, rabbit, dog and man. B-FN accumulates around neovascular structures in aggressive tumours and other tissues undergoing angiogenesis, such as the endometrium in the proliferative phase and some ocular structures in pathological conditions, but is otherwise undetectable in normal adult tissues.

Tenascin-C is a large hexameric glycoprotein of the extracellular matrix which modulates cellular adhesion. It is involved in processes such as cell proliferation and cell migration and is associated with changes in tissue architecture as occurring during morphogenesis and embryogenesis as well as under tumourigenesis or angiogenesis. Several isoforms of tenascin-C can be generated as a result of alternative splicing which may lead to the inclusion of (multiple) domains in the central part of this protein, ranging from domain A1 to domain D (Borsi L et al Int J Cancer 1992; 52:688-692, Carnemolla B et al. Eur J Biochem 1992; 205:561-567, WO2006/050834). A specific binding member, as referred to herein, may bind tenascin-C. A specific binding member may bind tenascin-C domain A1.

Cancer, as referred to herein, may be a cancer which expresses, or has been shown to express, the ED-A isoform of fibronectin, the ED-B isoform of fibronectin and/or alternatively spliced tenascin C. Preferably the cancer expresses the ED-A isoform of fibronectin. For example, the cancer may be any type of solid or non-solid cancer or malignant lymphoma and especially germ cell cancer (such as teratocarcinoma), liver cancer, lymphoma (such as Hodgkin's or non-Hodgkin's lymphoma), leukaemia (e.g. acute myeloid leukaemia), sarcomas, skin cancer, melanoma, sarcoma, bladder cancer, breast cancer, uterine cancer, ovarian cancer, prostate cancer, lung cancer, colorectal cancer, cervical cancer, head and neck cancer, oesophageal cancer, pancreatic cancer, renal cancer, stomach cancer and cerebral cancer. Cancers may be familial or sporadic. Cancers may be metastatic or non-metastatic.

An inflammatory autoimmune disease, as referred to herein, may be an inflammatory autoimmune disease which is characterised by, or has been shown to be characterised by, expression of the ED-A isoform of fibronectin, the ED-B isoform of fibronectin and/or tenascin C. The conjugate used in the treatment of an inflammatory autoimmune disease, or delivery of IL4 to sites of inflammatory autoimmune disease in a patient, may be selected based on the expression of the ED-A isoform of fibronectin, ED-B isoform of fibronectin and tenascin C in said inflammatory autoimmune disease. Preferably, the inflammatory autoimmune disease is selected from the group consisting of: rheumatoid arthritis (RA), multiple sclerosis (MS), endometriosis, autoimmune diabetes (such as diabetes mellitus type 1), inflammatory bowel disease (IBD), psoriasis, psoriatic arthritis, Behçet's disease and periodontitis. More preferably, the autoimmune disease is selected from the group consisting of: rheumatoid arthritis (RA), multiple sclerosis (MS), Behçet's disease, endometriosis, autoimmune diabetes (such as diabetes mellitus type 1), and psoriasis.

Rheumatoid arthritis (RA) is an autoimmune disease that may result in a chronic, systemic inflammatory disorder that may affect many tissues and organs, but principally attacks flexible (synovial) joints.

Psoriasis is an autoimmune disease that may result in a chronic systemic inflammatory disorder that may affect any part of the body but is most commonly found on the elbows, knees, lower back and scalp. Psoriasis may result in red, flaky patches of skin covered with silvery scales.

Psoriatic arthritis is an autoimmune disease which causes inflammation and pain in the joints, although other parts of the body may also be affected. Psoriatic arthritis is a type of inflammatory arthritis and is often associated with psoriasis.

Endometriosis is a gynaecological disease in which cells from the endometrium grow outside the uterine cavity. Symptoms of endometriosis include pelvic pain and fertility problems. Endometriosis, as referred to herein, may be Stage I, Stage II, Stage III, and/or Stage IV endometriosis according to the Revised Classification of the American Society of Reproductive Medicine, 1996, Fertility and Sterility 67 (5): 817-21.

Behçet's disease is an immune-mediated small-vessel systemic vasculitis that often presents with mucous membrane ulceration and ocular problems.

Multiple sclerosis is an inflammatory disease in which the fatty myelin sheaths around the axons of the brain and spinal cord are damaged, leading to demyelination and scarring. “Multiple sclerosis”, as referred to herein, may refer to relapsing remitting, secondary progressive, primary progressive, and/or progressive relapsing multiple sclerosis.

Autoimmune Insulitis refers to a lymphocytic infiltration of the islets of Langerhans of the pancreas. Autoimmune Insulitis is frequently associated with new-onset type 1 diabetes mellitus.

Autoimmune diabetes is a form of diabetes mellitus that results from autoimmune destruction of the insulin-producing islets of Langerhans of the pancreas. Autoimmune disease diabetes can occur in both adults and children. Autoimmune diabetes, as referred to herein, is preferably diabetes mellitus type 1.

Inflammatory Bowel Disease is a group of inflammatory conditions that affect the colon and small intestine. The major types of IBD are Crohn's disease (CD) and ulcerative colitis (UC), while other types of IBD include collagenous colitis, lymphocytic colitis, ischaemic colitis, diversion colitis, Behçet's disease and indeterminate colitis. CD can affect any part of the gastrointestinal tract, whereas UC is typically restricted to the colon and rectum.

IBD, as referred to herein, may be CD, UC, collagenous colitis, lymphocytic colitis, ischaemic colitis, diversion colitis, Behçet's disease or indeterminate colitis. In particular, the terms CD, UC, collagenous colitis, lymphocytic colitis, ischaemic colitis, diversion colitis, Behçet's disease and indeterminate colitis, as used herein, may refer to active CD, active UC, active collagenous colitis, active lymphocytic colitis, active ischaemic colitis, active diversion colitis, and active indeterminate colitis, respectively. In one embodiment, the IBD may be CD or UC.

Other aspects and embodiments of the invention provide the aspects and embodiments described above with the term “comprising” replaced by the term “consisting of” and the aspects and embodiments described above with the term “comprising” replaced by the term “consisting essentially of”.

It is to be understood that the application discloses all combinations of any of the above aspects and embodiments described above with each other, unless the context demands otherwise. Similarly, the application discloses all combinations of the preferred and/or optional features either singly or together with any of the other aspects, unless the context demands otherwise.

Modifications of the above embodiments, further embodiments and modifications thereof will be apparent to those skilled in the art given the present disclosure including the following experimental exemplification.

All documents mentioned in this specification are incorporated herein by reference in their entirety for all purposes.

“and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein.

Unless context dictates otherwise, the descriptions and definitions of the features set out above are not limited to any particular aspect or embodiment of the invention and apply equally to all aspects and embodiments which are described.

Certain aspects and embodiments of the invention will now be illustrated by way of example and with reference to the figures described above.

### EXAMPLES

The present examples describe the construction of antibody-IL4 conjugates and demonstrate the suitability of such conjugates for targeting cancer in vivo.

### Examples Relating to the Preparation and In Vitro Characterisation of Antibody-(Human) IL4 Conjugates

### Example 1—Cloning of ScDbF8-hIL4, ScFvF8-hIL4-ScFvF8 and hIL4-ScDbF8

Three conjugates containing human IL4 and the antibody F8 (Villa et al., 2008) (specific to the EDA domain of fibronectin, a marker of tumour angiogenesis) are termed (i) ScDbF8-hIL4, (ii) ScFvF8-hIL4-ScFvF8 and (iii) hIL4-ScDbF8.

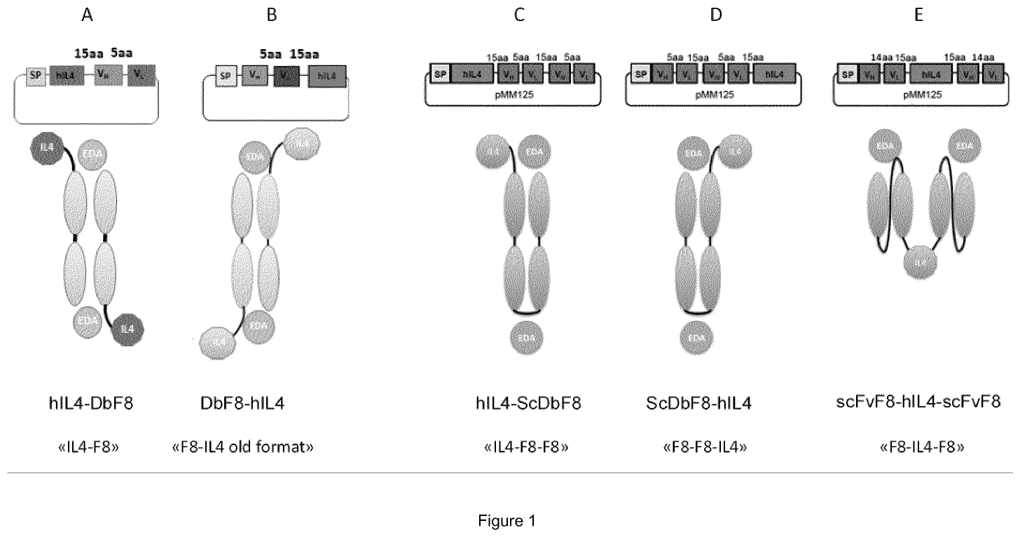

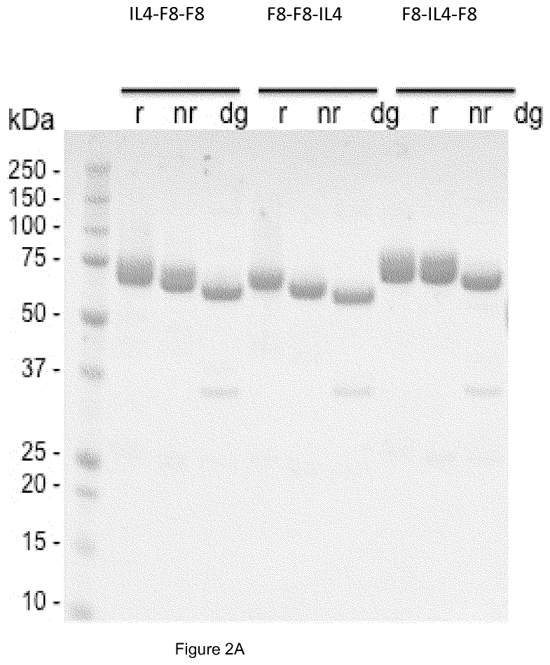

A schematic illustration of the molecular formats is presented in FIG. 1.

The genes encoding the three conjugates above were generated using PCR assembly. For ScDbF8-hIL4 and hIL4-ScDbF8, the sequence encoding human IL4 (lacking the signal peptide sequence) was linked via a sequence encoding a 15 amino acid glycine-serine-linker [(G₄S)₃] as shown in SEQ ID NO: 34 either to the C-terminus (in ScDbF8-hIL4) or the N-terminus (hIL4-ScDbF8) of the gene encoding the F8 antibody in single chain diabody format (ScDb, two antibody fragments fused by a [(S₄G)₃] linker as shown in SEQ ID NO: 33, each consisting of heavy chain and light chain connected via a GGSGG-linker (SEQ ID NO: 39). The amino acid sequences of hIL4-ScDbF8 is shown in SEQ ID NO: 37 and the amino acid sequences of ScDbF8-hIL4 is shown in SEQ ID NO: 38.

A sequence encoding an IgG-derived signal peptide was added at the N-terminus to enable high yield production of the encoded fusion proteins. Using engineered HindIII and NotI restriction sites, the genes were cloned into the pMM125 mammalian cell expression vector.

For ScFvF8-hIL4-ScFvF8, the sequence encoding human IL4 was fused between two genes encoding F8 antibody in single chain variable fragment (scFv; consisting of heavy chain and light chain connected via a 14 amino acid glycine-serine-linker as shown in SEQ ID NO: 40), by means of a 15 amino acid serine-glycine-linker [(S₄G)₃)] linker (SEQ ID NO: 33). A sequence encoding an IgG-derived signal peptide was added at the N-terminus to enable high yield production of the encoded fusion proteins. Using engineered HindIII and NotI restriction sites, the genes were cloned into the pMM125 mammalian cell expression vector. The sequences of the genes used to encode the hIL4-ScDbF8, ScDbF8-hIL4, and scFvF8-hIL4-scFvF8 fusion proteins are shown in SEQ ID NOs: 41, 42 and 43, respectively, while the amino acid sequences of the mature hIL4-ScDbF8, ScDbF8-hIL4, and scFvF8-hIL4-scFvF8 fusion proteins employed in the experiments reported below are shown in SEQ ID NOs: 37, 38 and 35, respectively. The signal peptides are cleaved after expression of the fusion proteins and thus are not part of the mature fusion proteins

### Example 2—Expression of ScDbF8-hIL4, ScFvF8-hIL4-ScFvF8 and hIL4-ScDbF8

Fusion proteins comprising human IL4 were produced by transient gene expression in suspension adapted CHO-S cell cultures. Following transfection cells were maintained in PowerCHO-2 medium (supplemented with 4 mM Ultraglutamine), for 6 days at 31° C. under shaking conditions, after which the culture supernatant was harvest by centrifugation and further processed to purify the fusion protein.

### Purification of Fusion Proteins Using Protein a Resin

Transfected CHO-S cell suspension cultures were centrifuged for 30 minutes at 5000 rpm at 4° C. The supernatant was further clarified by filtration using 0.45 um filters (rapid Flow Bottle Top filters, Nalgene). Protein A resin (Ultra linked Protein A resin, Sino Biological Inc.) was added to the filtered supernatant and the mixture incubated under shaking conditions for ca 1 h. The resin was than collected into a liquid chromatography column (SIGMA), and washed with “buffer A” (100 mM NaCl, 0.5 mM EDTA, 0.1% Tween 20 in PBS pH 7.4) followed by a second wash with “buffer B” (500 mM NaCl, 0.5 mM EDTA in PBS pH 7.4). The fusion proteins comprising human IL4 were eluted by gravity flow using 0.1 M glycine, pH 3. Aliquots were collected and fractions containing the fusion protein, as confirmed by UV spectrometry, were pooled and dialysed overnight against PBS.

### Deglycosylation of Fusion Proteins

Deglycosylation of fusion proteins comprising human IL4 was performed using PNGase F (NEB) to remove complex oligosaccharides from N-linked glycoproteins. Under native conditions ca 2-5 μg of fusion protein were diluted in Glycobuffer 2 (NEB), and after addition of 1 μl of PNGaseF (500 U, NEB) the reaction mix was incubated over night at 37° C. Afterwards all samples were analyzed by SDS-PAGE. The effect of deglycosylation is visible as mobility shift and sharpening of bands in SDS-PAGE gels.

### Size Exclusion Chromatography of Fusion Proteins

Size exclusion chromatography of fusion proteins was performed using a Superdex 200 increase 10/300 GL column (GE Healthcare) with PBS as running buffer on an AKTA-FPLC system (GE healthcare). 100 μl protein solutions were injected into a loop and automatically injected onto the column. UV absorbance at 280 nm was assessed over time.

### Results

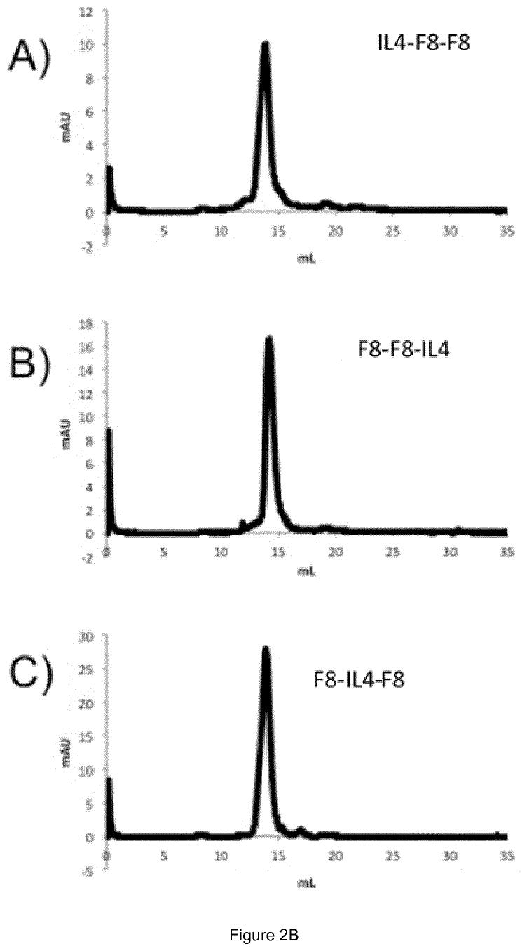

The purified fusion proteins exhibited favourable biochemical properties as confirmed using (1) SDS-PAGE and (2) size exclusion chromatography. SDS-PAGE analysis using coomassie staining revealed broad protein bands slightly higher than the estimated sizes of ca 66.7 kDa for hIL4-ScDbF8 and ScDbF8-hIL4, and 68 kDa for F8-hIL4-F8, respectively. This shift was caused by the presence of N-linked glycans, which could be removed using PNGase F, leading to a band shift to the expected size for the fusion proteins (FIG. 2A). Size exclusion chromatography analysis further confirmed the homogeneity of the conjugate preparations (FIG. 2B).

### Example 3—Characterization of ScDbF8-hIL4, ScFvF8-hIL4-ScFvF8 and hIL4-ScDbF8

### Biacore Analysis

Using surface plasmon resonance (Biacore 3000 system, GE Healthcare) the binding affinity to ED-A of the fusion proteins was analysed. A microsensor chip (CM5, GE Healthcare) was coated with 11A12, a recombinantly expressed ED-A, with ca 1500 resonance units coating density. For analysis on surface plasmon resonance, proteins were filtered with a syringe driven filter unit (Millex®-GV, Low protein binding durapore membrane, 0.22 μm) and their concentration determined with a spectrophotometer (NanoDrop 2000, Witec ag).

### ELISA Test

The binding capacity of the fusion proteins were further confirmed by ELISA. Recombinant EDA-domain was immobilized on maxisorp wells (Nunc-lmmuno) over night at room temperature. On the day of binding assessment, wells were blocked using 200 μl of blocking solution (1% Casein hydrolysate, 0.05% Tween-20, 0.02% NaN₃, in PBS pH7.4) for 2 hours at room temperature. Wells were washed three times and the primary antibody solution (anti-hIL4, eBioscience, 1:1000 in blocking buffer) was added. Plates were incubated for 1 h at RT. Afterwards wells were washed with three times with PBS and the secondary antibody was added (anti-mouse IgG-HRP, Sigma, 1:1000 in blocking buffer). After 1 h incubation at room temperature, plates were washed three times with 0.1% PBS-Tween and PBS alone after which the POD substrate (Roche) was added. The reaction was stopped using 1M H₂SO₄ and the read out was obtained measuring absorption at 405 nm and 650 nm using a UV spectrophotometer (LEDETECT 96 microplate, Dynamica Scientific Ltd.).

### Bioactivity Assay for IL4

TF-1 cells (SIGMA) were cultured in Complete Medium (RPMI 1640 medium, 1 mM sodium pyruvate, 10% FCS, 5 ng/mL recombinant human GM-CSF). The TF-1 proliferation assay was performed in 96 wells microtiter plates by incubating 40,000 cells per well in the presence of several doses of either recombinant human IL4 (eBioscience), and corresponding hIL4-F8 fusion proteins ranging between 2.7 and 0.02 nM. Unstimulated cells were used as negative control.

A MTS Cell proliferation assay (CellTiter-96® AQueousOne Solution (Promega) was employed to evaluate cell response: after a 72-hour culture period, 20 μL MTS solution was added to each well for an additional 4 hours incubation. The intensity of each colorimetric signal was measured at 490 nm (vs. 640 nm) in a microtiter plate reader, 16 hours (overnight) following addition of substrate reagent.

### Results

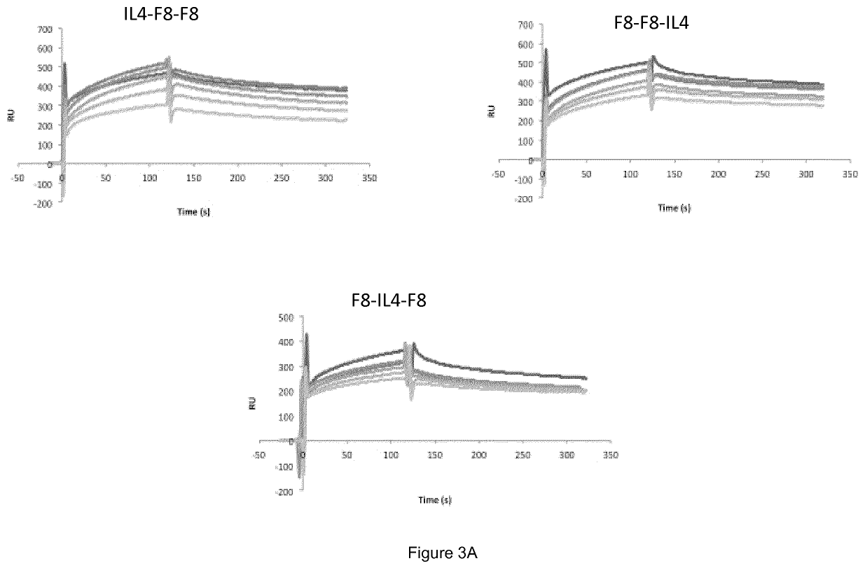

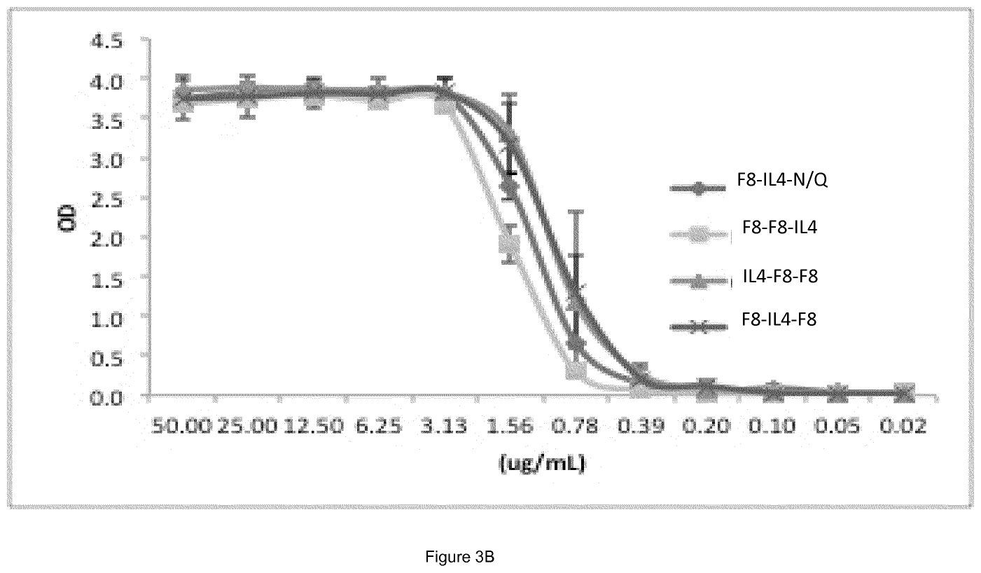

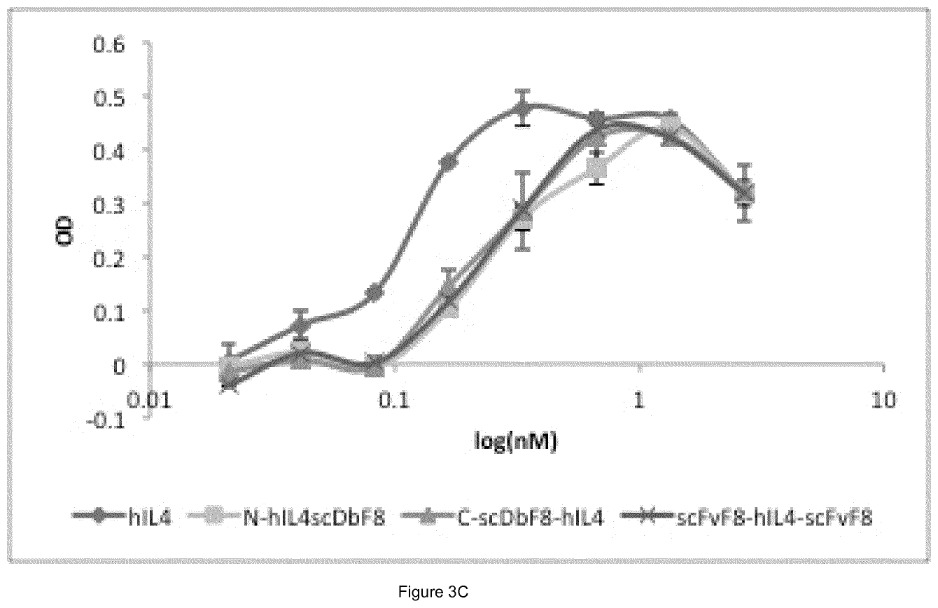

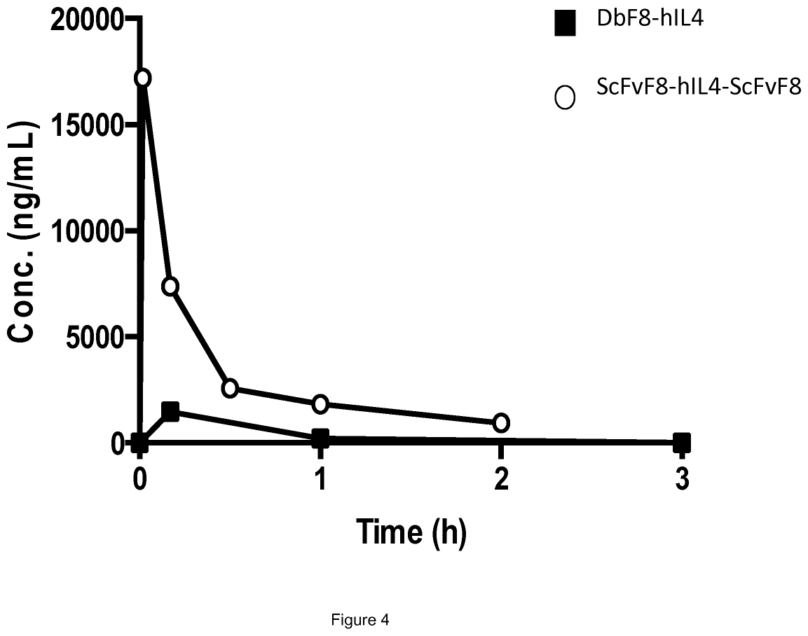

After fusion with human IL4, the binding capacity of the F8 moiety to the ED-A of fibronectin was maintained, as confirmed using surface plasmon resonance (Biacore) (FIG. 3A) and ELISA analysis (FIG. 3B).

Finally, the human IL4 also retained its biological activity after fusion with the F8 antibody in the ScDbF8-IL4, ScFvF8-IL4-ScFvF8 and IL4-ScDbF8 fusion proteins, as determined using a TF-1 proliferation assay (FIG. 3C).

### Examples Relating to the In Vivo Characterisation of Antibody-(Human) IL4 Conjugates

### Example 4—Biodistribution Studies

We compared in biodistribution studies five different IL4 fusion proteins, namely the three new fusion proteins (i) ScDbF8-hIL4, (ii) ScFvF8-hIL4-ScFvF8 (iii) hIL4-ScDbF8 and two additional IL4 fusion proteins based on the conjugation of human IL4 to a diabody as described in WO2014/173570. These two additional fusion proteins are named (iv) DbF8-hIL4 and (v) hIL4-DbF8.

For radiolabelling purposes, the five fusion proteins DbF8-hIL4, hIL4-DbF8, ScDbF8-hIL4, ScFvF8-hIL4-ScFvF8 and hIL4-ScDbF8 were purified over size exclusion chromatography and then radioiodinated with Iodine 125. A total of 10-80 μg of the fusion protein preparation were injected into the tail vein of immunocompetent 129Sv mice bearing subcutaneously implanted F9 murine teratocarcinomas. Mice were sacrificed 24 h after injection. Organs were weighed and radioactivity was counted with a Packard Cobra gamma counter.

### Results

Table 1 shows that the cumulative tumor to organ ratio, was superior for the ScFv-hIL4-ScFv as compared to the other protein formats.

TABLE 1
Tumor/organ ratio

| | ScFvF8-hIL4- | ScDbF8- | hIL4- | DbF8- | hIL4- |
|---|---|---|---|---|---|
| | ScFvF8 | hIL4 | ScDbF8 | hIL4 | DbF8 |
| liver | 2.21 | 0.75 | 1.25 | 2.21 | 4.97 |
| lung | 3.68 | 2.52 | 3.29 | 1.91 | 3.61 |
| heart | 6.97 | 3.61 | 5.98 | 3.54 | 5.77 |
| kidney | 1.42 | 0.63 | 0.69 | 0.97 | 2.98 |
| blood | 8.43 | 4.99 | 6.58 | 1.08 | 1.54 |
| TOTAL | 22.71 | 12.50 | 17.79 | 9.71 | 18.87 |

### Example 5—Pharmacokinetic Analysis in Monkeys

Cynomolgus monkeys (Macaca fasciculiaris) in the weight range 2 to 4 kg and greater than 18 months of age were used for a pharmacokinetic study in which they were administered 0.37 mg/kg of either DbF8-hIL4 or ScFvF8-hIL4-ScFvF8. Animals received one intravenous dose administered as a bolus injection via a cephalic vein or other superficial vein. Blood samples were taken from all animals for pharmacocokinetic analysis following the administration of the test agent. The samples (2.5 mL) were collected from the femoral vein/artery into plain SST tubes and placed into uniquely labelled polypropylene tubes. The blood samples (2.5 mL) were collected from the femoral vein/artery and centrifuged (10 min, 2300 g, 20° C. nominal). The resultant plasma was frozen and stored at >−50° C. prior to despatch for analysis.

Pharmacokinetic (PK) analysis was performed using an ELISA method with the 11-EDA-12 antigen for plate coating and a mouse anti-IL4 antibody (primary antibody) followed by an anti-mouse IgG HRP antibody (secondary antibody), as detection components. If the molecule was present in a sample, the addition of the substrate produced a detectable signal, proportional to the concentration of the test item in the sample.

### Results

When compared to DbF8-hIL4, the novel format ScFvF8-hIL4-ScFvF8 shows a superior pharmacokinetic profile characterized by a longer half-life (FIG. 4).

### Example 6—Comparative Experiments with Prior Art Immunoconjugates

Both ScFvF8-mIL4-ScFvF8 and ScFvF8-mIL7-ScFvF8 (Pasche et al. 2011), were cloned into pcDNA3.1 and expressed by transient gene expression in CHO-S cells (as previously reported in literature, Rajendra Y et al, 2011).

On the day of transfection, 1.2×10⁷ CHO-S cells in suspension were centrifuged and resuspended in 300 mL ProCHO4 (Lonza), supplemented with 4 mM Ultra-Glutamine (Lonza). 750 μg of plasmid DNA, encoding for the gene of interest, and 3 mL of 25 kDa linear polyethyleneimine (PEI) (1 mg/mL solution, Polysciences, Germany) were then added to the cells. The transfected cultures were incubated in a shaker incubator at 31° C. for 6 days. The fusion proteins were purified from the cell culture medium by Protein A affinity chromatography (Protein A from Sino Biologicals, elution buffer Glycine 0.1M pH 3.00). After elution, the protein was desalted and stored in PBS (30 mM Na2HPO4, 20 mM NaH2PO4, 100 mM NaCl, pH 7.4).

After radioiodination with ¹²⁵I, a total of 10 μg of radiolabelled protein (named “HOT”) with 33 μg of NON-radiolabeled fusion protein (named “COLD”) were injected into the tail vein of BALB/c nude mice bearing subcutaneously implanted A431 human squamous carcinoma. Mice were sacrificed 24 h after injection. Organs were weighed and radioactivity was counted with a Packard Cobra gamma counter. The radioactive content of representative organs was recorded and expressed as percentage of injected dose over gram of tissue (% ID/g).

### Results

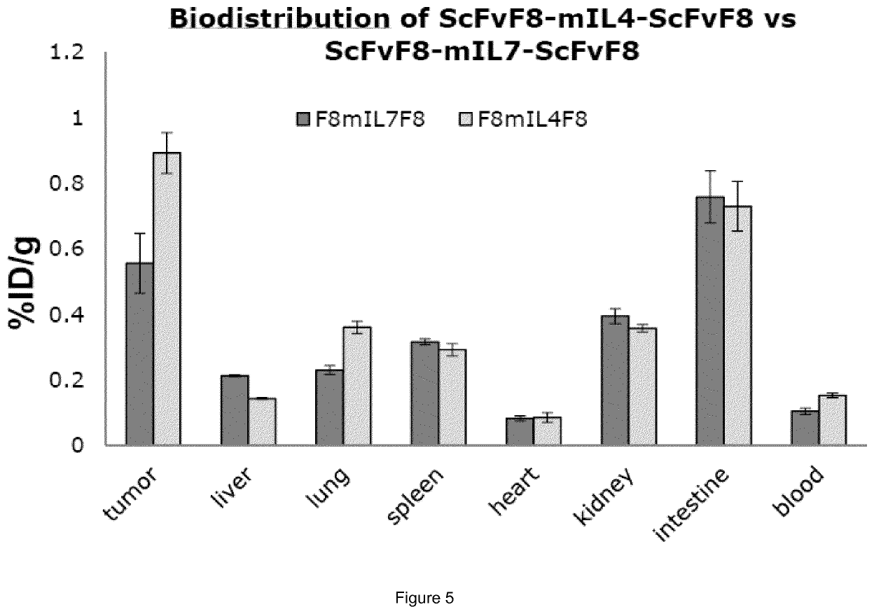

ScFvF8-mIL4-ScFvF8 and ScFvF8-mIL7-ScFvF8 (Pasche (2011) are two immunocytokines which target the extra-domain A of human oncofoetal fibronectin (EDA). Both fusion proteins consist of a sequential fusion of scFvF8 with a murine interleukin (either mIL7 or mIL4) followed by a second scFvF8 moiety. The goal of this experiment was to compare the targeting performance of these two immunocytokines. Biodistribution studies in A431 human carcinoma mouse model revealed that surprisingly the tumor accumulation of ScFvF8-mIL4-ScFvF8 (0.89% ID/g) is much higher than the tumor accumulation of ScFvF8-mIL7-ScFvF8 (0.55% ID/g), while the uptake of the two immunocytokines in normal organs is comparable (FIG. 5). Taken together, these data prove the superiority of ScFvF8-mIL4-ScFvF8 in terms of both tumor to organ, and tumor to blood ratio.

### REFERENCES

Amit A G, Mariuzza R A, Phillips S E, Poljak R J., Three-dimensional structure of an antigen-antibody complex at 2.8 A resolution. 1986; Science, 233: 747-753.
Andersen et al. Recombinant protein expression for therapeutic applications. Current Opinion in Biotechnology, 2002; 13: 117-23.
Altschul et al. Basic local alignment search tool. J. Mol. Biol. 1990; 215: 405-410.
Altschul S F, Madden T L, Schäffer A A, Zhang J, Zhang Z, Miller W, Lipman D J., Gapped BLAST and PSI-BLAST: a new generation of protein database search programs. Nucl. Acids Res., 1997; 25: 3389-3402.
Ausubel et al. (1999) 4th eds., Short Protocols in Molecular Biology: A Compendium of Methods from Current Protocols in Molecular Biology, John Wiley & Sons.
Bagshawe et al., Antibody, Immunoconjugates and Radiopharmaceuticals, 1991; 4: 915-922.
Bird R E, Hardman K D, Jacobson J W, Johnson S, Kaufman B M, Lee S M, Lee T, Pope S H, Riordan G S, Whitlow M., Single-chain antigen-binding proteins. Science, 1988; 242: 423-426.
Borsi et al., Monoclonal antibodies in the analysis of fibronectin isoforms generated by alternative splicing of mRNA precursors in normal and transformed human cells. J. Cell. Biol., 1987; 104, 595-600.
Borsi L, Carnemolla B, Nicolò G, Spina B, Tanara G, Zardi L., Expression of different tenascin isoforms in normal, hyperplastic and neoplastic human breast tissues. Int J Cancer 1992; 52:688-692.
Borsi L, Balza E, Carnemolla B, Sassi F, Castellani P, Berndt A, et al. Selective targeted delivery of TNFalpha to tumor blood vessels. Blood. 2003; 102: 4384-92.
Brack S S, Silacci M, Birchler M, Neri D. Tumor-targeting properties of novel antibodies specific to the large isoform of tenascin-C. Clin Cancer Res. 2006; 12:3200-8.
Carnemolla B et al., Comparison of human tenascin expression in normal, simian-virus-40-transformed and tumor-derived cell lines. Eur J Biochem, 1992; 205: 561-567.
Carnemolla B, Borsi L, Balza E, Castellani P, Meazza R, Berndt A, et al. Enhancement of the antitumor properties of interleukin-2 by its targeted delivery to the tumor blood vessel extracellular matrix. Blood. 2002; 99: 1659-65.
Caton A J, Herlyn D, Ross A H, Koprowski H., Identical D region sequences expressed by murine monoclonal antibodies specific for a human tumor-associated antigen. J. Immunol.; 1990, 144:1965-1968.
Chadd H E and Chamow S M., Therapeutic antibody expression technology. Current Opinion in Biotechnology, 2001; 12: 188-194.
Chomarat P, Banchereau J. An update on interleukin-4 and its receptor. Eur Cytokine Netw. 1997; 8: 333-44.
Chothia C and Lesk A M., Canonical structures for the hypervariable regions of immunoglobulins. J. Mol. Biol.; 1987, 196: 901-917. Chothia C, Lesk A M, Tramontano A, Levitt M, Smith-Gill S J, Air G, Sheriff S, Padlan E A, Davies D,
Tulip W R, et al., Conformations of immunoglobulin hypervariable regions. Nature, 1989; 342: 877-883.
Dela Cruz, J. S., Huang, T. H., Penichet, M. L. & Morrison, S. L. Antibody-cytokine fusion proteins: innovative weapons in the war against cancer. Clin Exp Med 4, 57-64 (2004).
Ebbinghaus et al., Int. J. Cancer, 2005, 116, 304-313.
Eigentler T K, Weide B, de Braud F, Spitaleri G, Romanini A, Pflugfelder A, et al. A dose-escalation and signal-generating study of the immunocytokine L19-IL2 in combination with dacarbazine for the Therapy of patients with metastatic melanoma. Clin Cancer Res. 2011; 17: 7732-42.
Fort M, Lesley R, Davidson N, Menon S, Brombacher F, Leach M, Rennick D. IL-4 Exacerbates Disease in a Th1 Cell Transfer Model of Colitis. J. Immunol. 2001; 166:2793-2800.
Frey K, Schliemann C, Schwager K, Giavazzi R, Johannsen M, Neri D. The immunocytokine F8-IL2 improves the therapeutic performance of sunitinib in a mouse model of renal cell carcinoma. J Urol. 2010; 184: 2540-8.
Frey K, Zivanovic A, Schwager K, Neri D. Antibody-based targeting of interferon-alpha to the tumor neovasculature: a critical evaluation. Integr Biol (Camb). 2011; 3: 468-78.
Frey K, Zivanovic A, Schwager K, Neri D., Antibody-based targeting of interferon-alpha to the tumor neovasculature: a critical evaluation. Integr Biol (Camb). 2011 April; 3(4):468-78.
Fukushi J, Ono M, Morikawa W, Iwamoto Y, Kuwano M. The Activity of Soluble VCAM-1 in angiogenesis Stimulated by IL-4 and IL-13. J. Immunol. 2000; 165:2818-2823.
Glennie et al., Preparation and performance of bispecific F(ab′ gamma)2 antibody containing thioether-linked Fab′ gamma fragments. J. Immunol., 1987; 139: 2367-2375.
Haan and Maggos, BioCentury, 2004; 12(5): A1-A6.
Harlow and Lane, Antibodies: A Laboratory Manual, Cold Spring Harbor Laboratory, Cold Spring Harbor N.Y., pp. 726, 1988
Holliger P, Prospero T, Winter G., “Diabodies”: small bivalent and bispecific antibody fragments. Proc. Natl. Acad. Sci. USA, 1993a; 90 6444-6448
Holliger P and Winter G., Engineering bispecific antibodies. Current Opinion Biotechnol, 1993b; 4: 446-449.
Holliger and Winter, Diabodies: small bispecific antibody fragments. Cancer Immunol. Immunother, 1997; 45:128-130.
Holliger P and Bohlen H., Engineering antibodies for the clinic. Cancer and Metastasis Rev., 1999; 18: 411-419.
Holt L J, Herring C, Jespers L S, Woolven B P, Tomlinson I M., Domain antibodies: proteins for therapy. Trends in Biotechnology. 2003; 21: 484-490.
Hu S, Shively L, Raubitschek A, Sherman M, Williams L E, Wong J Y, Shively J E, Wu A M., Minibody: A novel engineered anti-carcinoembryonic antigen antibody fragment (single-chain Fv-CH3) which exhibits rapid, high-level targeting of xenografts. Cancer Res., 1996; 56: 3055-
Huston J S, Levinson D, Mudgett-Hunter M, Tai M S, Novotny J, Margolies M N, Ridge R J, Bruccoleri R E, Haber E, Crea R, et al., Protein engineering of antibody binding sites: recovery of specific activity in an anti-digoxin single-chain Fv analogue produced in Escherichia coli. PNAS USA, 1988; 85: 5879-5883.
Janeway C. Immunobiology: the immune system in health and disease. 6th ed. New York: Garland Science; 2005.
Joosten Leo A B, Erik Lubberts, Monique M A Helsen, Tore Saxne, Christina J J Coenen-de Roo, Dick Heinegård and Wim B van den Berg. Protection against cartilage and bone destruction by systemic interleukin-4 treatment in established murine type II collagen induced arthritis, Arthritis Research, Vol 1, No 1, 81-91 (1999).
Joosten, Leo A. B., Erik Lubberts, Patrick Durez, Monique M. A. Helsen, Marike J. M. Jacobs, Michel Goldman, and Wim B. Van Den Berg, Role of Interleukin-4 And Interleukin-10 In Murine Collagen-Induced Arthritis, Arthritis And Rheumatism, Vol. 40, No. 2, 249-260 (1997).
Kabat et al., Sequences of Proteins of Immunological Interest. 1987; 4th Edition. U S Department of Health and Human Services.
Kabat et al., Sequences of Proteins of Immunological Interest. 1991a; 5th Edition, U S Department of Health and Human Services, Public Service, NIH, Washington, and later editions.
Kabat E A, Wu T T, Identical V region amino acid sequences and segments of sequences in antibodies of different specificities. Relative contributions of V H and V L genes, minigenes, and complementarity-determining regions to binding of antibody-combining sites. J. Immunol., 1991b; 147:1709-1719.
Kaspar M, Trachsel E, Neri D. The antibody-mediated targeted delivery of interleukin-15 and G M-CSF to the tumor neovasculature inhibits tumor growth and metastasis. Cancer Res. 2007; 67:4940-8.
Kay, Winter & McCafferty (1996)
Knappik A, Ge L, Honegger A, Pack P, Fischer M, Wellnhofer G, Hoess A, Wölle J, Plückthun A, Virnekäs B., Fully synthetic human combinatorial antibody libraries (HuCAL) based on modular consensus frameworks and CDRs randomized with trinucleotides. J. Mol. Biol., 2000; 296: 57-86.
Koide A, Bailey C W, Huang X, Koide S. The fibronectin type III domain as a scaffold for novel binding proteins. Journal of Molecular Biology, 1998; 284: 1141-1151.
Kohler and Milstein, Continuous cultures of fused cells secreting antibody of predefined specificity. Nature, 1975; 256: 495-497.
Kontermann and Muller, Intracellular and cell surface displayed single-chain diabodies. J. Immunol. Methods, 1999; 226: 179-188.
Kontermann and Dubel, Antibody Engineering, Springer-Verlag New York, LLC; ISBN: 3540413545, 2001.
Kontermann R E. Antibody-cytokine fusion proteins. Arch Biochem Biophys. 2012; 526:194-205.
Kornblihtt A R, Vibe-Pedersen K, Baralle F E., Human fibronectin: cell specific alternative mRNA splicing generates polypeptide chains differing in the number of internal repeats. Nucleic Acids Res., 1984; 12: 5853-5868.
Krebs B, Rauchenberger R, Reiffert S, Rothe C, Tesar M, Thomassen E, Cao M, Dreier T, Fischer D, Höss A, Inge L, Knappik A, Marget M, Pack P, Meng X Q, Schier R, Söhlemann P, Winter J, Wölle J, Kretzschmar T., High-throughput generation and engineering of recombinant human antibodies. Journal of Immunological Methods, 2001; 254: 67-84.
Kurtz D M, Tschetter L K, Allred J B, Geyer S M, Kurtin P J, Putnam W D, et al. Subcutaneous interleukin-4 (IL-4) for relapsed and resistant non-Hodgkin lymphoma: a phase II trial in the North Central Cancer Treatment Group, NCCTG 91-78-51. Leuk Lymphoma. 2007; 48:1290-8. Larrick and Thomas, Producing proteins in transgenic plants and animals. Current Opinion in Biotechnology, 2001; 12: 411-418.
Ledermann J A, Begent R H, Massof C, Kelly A M, Adam T, Bagshawe K D., A phase-I study of repeated therapy with radiolabelled antibody to carcinoembryonic antigen using intermittent or 55 continuous administration of cyclosporin A to suppress the immune response. Int. J. Cancer, 1991; 47: 659-664.
Li Z, Chen L, Qin Z. Paradoxical roles of IL-4 in tumor immunity. Cell Mol Immunol. 2009; 6(6):415-422.
Li Z, Jiang J, Wang Z, Zhang J, Xiao M, Wang C, Lu Y, Qin Z. Endogenous interleukin-4 promotes tumor development by increasing tumor cell resistance to apoptosis. Cancer Res. 2008; 68(21):8687-8694.
McCafferty J, Griffiths A D., Winter G, Chiswell D J, Phage antibodies: filamentous phage displaying antibody variable domains. Nature, 1990; 348, 552-554.
Mendez M J, Green L L, Corvalan J R, Jia X C, Maynard-Currie C E, Yang X D, Gallo M L, Louie D M, Lee D V, Erickson K L, Luna J, Roy C M, Abderrahim H, Kirschenbaum F, Noguchi M, Smith D H, Fukushima A, Hales J F, Klapholz S, Finer M H, Davis C G, Zsebo K M, Jakobovits A., Functional transplant of megabase human immunoglobulin loci recapitulates human antibody response in mice. Nature Genet, 1997; 15: 146-156.
Merchant, A. M., Z. Zhu, An efficient route to human bispecific IgG. Nature Biotech., 1998; 16: 677-681
Neri, D. & Bicknell, R. Tumour vascular targeting. Nat Rev Cancer 5, 436-446 (2005).
Nettelbeck D M, Miller D W, Jéröme V, Zuzarte M, Watkins S J, Hawkins R E, Miller R, Kontermann R E, Targeting of adenovirus to endothelial cells by a bispecific single-chain diabody directed against the adenovirus fiber knob domain and human endoglin (CD105). Molecular Therapy, 2001; 3, 882-891.
Nygren P A and Uhl6n M., Scaffolds for engineering novel binding sites in proteins. Current Opinion in Structural Biology, 1997; 7: 463-469.
Paolella G, Henchcliffe C, Sebastio G, Baralle F E., Sequence analysis and in vivo expression show that alternative splicing of E D-B and E D-A regions of the human fibronectin gene are independent events. Nucleic Acids Res., 1988; 16: 3545-3557.
Papadia F, Basso V, Patuzzo R, Maurichi A, Di Florio A, Zardi L, et al. Isolated limb perfusion with the tumor-targeting human monoclonal antibody-cytokine fusion protein L19-TNF plus melphalan and mild hyperthermia in patients with locally advanced extremity melanoma. J Surg Oncol. 2012.
Pasche N, Frey K, Neri D. The targeted delivery of IL17 to the mouse tumor neo-vasculature enhances angiogenesis but does not reduce tumor growth rate. Angiogenesis. 2012; 15:165-9.
Pasche N, Neri D. Immunocytokines: a novel class of potent armed antibodies. Drug Discov Today. 2012; 17:583-90.
Pasche N, Woytschak J, Wulhfard S, Villa A, Frey K, Neri D. Cloning and characterization of novel tumor-targeting immunocytokines based on murine IL7. J Biotechnol. 2011; 154:84-92.
Pasche N, Wulhfard S, Pretto F, Carugati E, Neri D. The antibody-based delivery of interleukin-12 to the tumor neovasculature eradicates murine models of cancer in combination with Paclitaxel. Clin Cancer Res. 2012; 18:4092-103.
Pearson W R, Lipman D J, Improved tools for biological sequence comparison. Proc Natl Acad Sci USA, 1988; 85: 2444-2448.
Pedretti M, Soltermann A, Arni S, Weder W, Neri D, Hillinger S. Comparative immunohistochemistry of L19 and F16 in non-small cell lung cancer and mesothelioma: two human antibodies investigated in clinical trials in patients with cancer. Lung Cancer. 2009; 64:28-33.
Plückthun A., Antibody engineering: advances from the use of Escherichia coli expression systems. Bio/Technology, 1991; 9: 545-551
Plückthun A., The Pharmacology of Monoclonal Antibodies, vol. 113 Rosenburg and Moore eds., Springer-Verlag, New York, 1994; pp. 269-3 15.
Reisfeld, R. A., Becker, J. C. & Gillies, S. D. Immunocytokines: a new approach to immunotherapy of melanoma. Melanoma Res 7 Suppl 2, S99-106 (1997).
Reiter et al., Engineering antibody Fv fragments for cancer detection and therapy: disulfide-stabilized Fv fragments. Nature Biotechnol., 1996; 14: 1239-1245.
Repp et al., J. Hemat., 1995; 377-382.
Revised American Society for Reproductive Medicine classification of endometriosis: 1996. Fertility and Sterility, 1997; 67: 817-21.
Ridgeway J B, Presta L G, Carter P., ‘Knobs-into-holes' engineering of antibody CH3 domains for heavy chain heterodimerization. Protein Eng., 1996; 9: 617-621.
Robinson, Sustained and Controlled Release Drug Delivery Systems, Marcel Dekker, Inc., New York, 1978.
Sambrook & Russell, Molecular Cloning: a Laboratory Manual: 3rd edition, Cold Spring Harbor Laboratory Press, 2001.
Savage, P., So, A., Spooner, R. A. & Epenetos, A. A. A recombinant single chain antibody interleukin-2 fusion protein. Br J Cancer 67, 304-310 (1993).
Segal et al., The three-dimensional structure of a phosphorylcholine-binding mouse immunoglobulin Fab and the nature of the antigen binding site. Proc Natl Acad Sci USA, 1974; 71: 4298-4302.
Sharon J, Structural correlates of high antibody affinity: three engineered amino acid substitutions can increase the affinity of an anti-p-azophenylarsonate antibody 200-fold. Proc Natl Acad Sci USA, 1990a; 87: 4814-4817.
Sharon J., Structural characterization of idiotopes by using antibody variants generated by site-directed mutagenesis. J. Immunol., 1990b; 144: 4863-4869.
Schliemann C, Palumbo A, Zuberbuhler K, Villa A, Kaspar M, Trachsel E, et al. Complete eradication of human B-cell lymphoma xenografts using rituximab in combination with the immunocytokine L19-IL2. Blood. 2009; 113:2275-83.
Schliemann C, Wiedmer A, Pedretti M, Szczepanowski M, Klapper W, Neri D. Three clinical-stage tumor targeting antibodies reveal differential expression of oncofetal fibronectin and tenascin-C isoforms in human lymphoma. Leuk Res. 2009; 33:1718-22.
Schrama, D., Reisfeld, R. A. & Becker, J. C. Antibody targeted drugs as cancer therapeutics. Nat Rev Drug Discov 5, 147-159 (2006).
Schwager K, Kaspar M, Bootz F, Marcolongo R, Paresce E, Neri D, Trachsel E. Preclinical characterization of DEKAVIL (F8-IL10), a novel clinical-stage immunocytokine which inhibits the progression of collagen-induced arthritis. Arthritis Research & Therapy 2009, 11(5): R142.
Schwager K, Kaspar M, Bootz F, Marcolongo R, Paresce E, Neri D, et al. Preclinical characterization of DEKAVIL (F8-IL10), a novel clinical-stage immunocytokine which inhibits the progression of collagen-induced arthritis. Arthritis Res Ther. 2009; 11: R142.
Schwager K, Bootz F, Imesch P, Kaspar M, Trachsel E, Neri D. The antibody-mediated targeted delivery of interleukin-10 inhibits endometriosis in a syngeneic mouse model. Hum Reprod. 2011; 26: 2344-52.
Schwager K, Villa A, Rosli C, Neri D, Rosli-Khabas M, Moser G. A comparative immunofluorescence analysis of three clinical-stage antibodies in head and neck cancer. Head Neck Oncol. 2011; 3: 25. Smith and Waterman, J. Mol Biol., 1981; 147: 195-197.
Staerz and Bevan, Hybrid hybridoma producing a bispecific monoclonal antibody that can focus effector T-cell activity. Proc Natl Acad Sci USA, 1986; 83: 1453-7.
Suresh M R, Cuello A C, Milstein C., Bispecific monoclonal antibodies from hybrid hybridomas. Method. Enzymol., 1986; 121: 210-228.
Tepper R I, Pattengale P K, Leder P. Murine interleukin-4 displays potent anti-tumor activity in vivo. Cell. 1989; 57: 503-12.
Tepper R I. The tumor-cytokine transplantation assay and the antitumor activity of interleukin-4. Bone Marrow Transplant. 1992; 9 Suppl 1:177-81.
Villa A, Trachsel E, Kaspar M, Schliemann C, Sommavilla R, Rybak J N, et al. A high-affinity human monoclonal antibody specific to the alternatively spliced EDA domain of fibronectin efficiently targets tumor neo-vasculature in vivo. Int J Cancer. 2008; 122: 2405-13.
Viti F, Tarli L, Giovannoni L, Zardi L, Neri D. Increased binding affinity and valence of recombinant antibody fragments lead to improved targeting of tumoral angiogenesis. Cancer Res. 1999; 59:347-52.
Volpert O V, Fong T, Koch A E, Peterson J D, Waltenbaugh C, Tepper R I, et al. Inhibition of angiogenesis by interleukin 4. J Exp Med. 1998; 188: 1039-46.
Ward et al., Binding activities of a repertoire of single immunoglobulin variable domains secreted from Escherichia coli. Nature, 1989; 341: 544-546.
Walz M, Overbergh L, Mathieu C, Kolb H, Martin S. A Murine Interleukin-4-lg Fusion Protein Regulates the Expression of Th1- and Th2-Specific Cytokines in the Pancreas of NOD Mice. Horm. Metab. Res. 2002; 34:561-569.
Wei M X, Tamiya T, Hurford R K, Jr., Boviatsis E J, Tepper R I, Chiocca E A. Enhancement of interleukin-4-mediated tumor regression in athymic mice by in situ retroviral gene transfer. Hum Gene Ther. 1995; 6: 437-43.
Wess, BioCentury, The Bernstein Report on BioBusiness, 2004; 12(42), A1-A7.
Whitehead R P, Lew D, Flanigan R C, Weiss G R, Roy V, Glode M L, et al. Phase I I trial of recombinant human interleukin-4 in patients with advanced renal cell carcinoma: a southwest oncology group study. J Immunother. 2002; 25: 352-8.
Whitehead R P, Unger J M, Goodwin J W, Walker M J, Thompson J A, Flaherty L E, et al. Phase I I trial of recombinant human interleukin-4 in patients with disseminated malignant melanoma: a Southwest Oncology Group study. J Immunother. 1998; 21: 440-6.
Wiernik P H, Dutcher J P, Yao X, Venkatraj U, Falkson C I, Rowe J M, et al. Phase I I study of interleukin-4 in indolent B-cell non-Hodgkin lymphoma and B-cell chronic lymphocytic leukemia: a study of the Eastern Cooperative Oncology Group (E5Y92). J Immunother. 2010; 33: 1006-9.
Yu J S, Wei M X, Chiocca E A, Martuza R L, Tepper R I. Treatment of glioma by engineered interleukin 4-secreting cells. Cancer Res. 1993; 53: 3125-8.
Zamorano J, Wang H Y, Wang L M, Pierce J H, Keegan A D. IL-4 protects cells from apoptosis via the insulin receptor substrate pathway and a second independent signaling pathway. J Immunol. 1996; 157: 4926-34.

Sequence listing
Amino acid sequence of F8 VH
(SEQ ID NO: 1)
EVQLLESGGGLVQPGGSLRLSCAASGFTFSLFTMSWVRQAPGKGLEWVSAISGSGGSTYYADSVKGRFTISRDNS
KNTLYLQMNSLRAEDTAVYYCAKSTHLYLFDYWGQGTLVTVSS
Amino acid sequence of F8 VL
(SEQ ID NO: 2)
EIVLTQSPGTLSLSPGERATLSCRASQSVSMPFLAWYQQKPGQAPRLLIYGASSRATGIPDRFSGSGSGTDFTLT
ISRLEPEDFAVYYCQQMRGRPPTFGQGTKVEIK
Amino acid sequence of the F8 single chain Fv (ScFv)
In BOLD UNDERLINED is the fourteen amino acid linker between VH and VL of the
single chain Fv
(SEQ ID NO: 3)
EVQLLESGGGLVQPGGSLRLSCAASGFTFSLFTMSWVRQAPGKGLEWVSAISGSGGSTYYADSVKGRFTISRDNS
KNTLYLQMNSLRAEDTAVYYCAKSTHLYLFDYWGQGTLVTVSSGGGGSGGGGSGGGGEIVLTQSPGTLSLSPGER
ATLSCRASQSVSMPFLAWYQQKPGQAPRLLIYGASSRATGIPDRFSGSGSGTDFTLTISRLEPEDFAVYYCQQMR
GRPPTFGQGTKVEIK
Amino acid sequence of F8 diabody (Db)
In BOLD UNDERLINED is the five amino acid linker between VH and VL of the diabody
(SEQ ID NO: 4)
EVQLLESGGGLVQPGGSLRLSCAASGFTFSLFTMSWVRQAPGKGLEWVSAISGSGGSTYYADSVKGRFTISRDNS
KNTLYLQMNSLRAEDTAVYYCAKSTHLYLFDYWGQGTLVTVSSGGSGGEIVLTQSPGTLSLSPGERATLSCRASQ
SVSMPFLAWYQQKPGQAPRLLIYGASSRATGIPDRFSGSGSGTDFTLTISRLEPEDFAVYYCQQMRGRPPTFGQG
TKVEIK
Amino acid sequences of F8 CDR's
F8 CDR1 VH
(SEQ ID NO: 5)
LFT
F8 CDR2 VH
(SEQ ID NO: 6)
SGSGGS
F8 CDR3 VH
(SEQ ID NO: 7)
STHLYL
F8 CDR1 VL
(SEQ ID NO: 8)
MPF
F8 CDR2 VL
(SEQ ID NO: 9)
GASSRAT
F8 CDR3 VL
(SEQ ID NO: 10)
MRGRPP
Amino acid sequence of L19 VH
(SEQ ID NO: 11)
EVQLLESGGGLVQPGGSLRLSCAASGFTFSSFSMSWVRQAPGKGLEWVSSISGSSGTTYYADSVKGRFTISRDNS
KNTLYLQMNSLRAEDTAVYYCAKPFPYFDYWGQGTLVTVSS
Amino acid sequence of L19 VL
(SEQ ID NO: 12)
EIVLTQSPGTLSLSPGERATLSCRASQSVSSSFLAWYQQKPGQAPRLLIYYASSRATGIPDRFSGSGSGTDFTLT
ISRLEPEDFAVYYCQQTGRIPPTFGQGTKVEIK
Amino acid sequence of the L19 single chain Fv (ScFv)
In BOLD UNDERLINED is the twelve amino acid linker between VH and VL of the
single chain Fv
(SEQ ID NO: 13)
EVQLLESGGGLVQPGGSLRLSCAASGFTFSSFSMSWVRQAPGKGLEWVSSISGSSGTTYYADSVKGRFTISRDNS
KNTLYLQMNSLRAEDTAVYYCAKPFPYFDYWGQGTLVTVSSGDGSSGGSGGASEIVLTQSPGTLSLSPGERATLS
CRASQSVSSSFLAWYQQKPGQAPRLLIYYASSRATGIPDRFSGSGSGTDFTLTISRLEPEDFAVYYCQQTGRIPP
TFGQGTKVEIK
Amino acid sequence of L19 diabody (Db)
In BOLD UNDERLINED is the five amino acid linker between VH and VL of the diabody
(SEQ ID NO: 14)
EVQLLESGGGLVQPGGSLRLSCAASGFTFSSFSMSWVRQAPGKGLEWVSSISGSSGTTYYADSVKGRFTISRDNS
KNTLYLQMNSLRAEDTAVYYCAKPFPYFDYWGQGTLVTVSSGGSGGEIVLTQSPGTLSLSPGERATLSCRASQSV
SSSFLAWYQQKPGQAPRLLIYYASSRATGIPDRFSGSGSGTDFTLTISRLEPEDFAVYYCQQTGRIPPTFGQGTK
VEIK
Amino acid sequences of L19 CDR's
L19 CDR1 VH
(SEQ ID NO: 15)
SFSMS
L19 CDR2 VH
(SEQ ID NO: 16)
SISGSSGTTYYADSVKG
L19 CDR3 VH
(SEQ ID NO: 17)
PFPYFDY
L19 CDR1 VL
(SEQ ID NO: 18)
RASQSVSSSFLA
L19 CDR2 VL
(SEQ ID NO: 19)
YASSRAT
L19 CDR3 VL
(SEQ ID NO: 20)
QQTGRIPPT
Amino acid sequence of F16 VH
(SEQ ID NO: 21)
EVQLLESGGGLVQPGGSLRLSCAASGFTFSRYGMSWVRQAPGKGLEWVSAISGSGGSTYYADSVKGRFTISRDNS
KNTLYLQMNSLRAEDTAVYYCAKAHNAFDYWGQGTLVTVSR
Amino acid sequence of F16 VL
(SEQ ID NO: 22)
SSELTQDPAVSVALGQTVRITCQGDSLRSYYASWYQQKPGQAPVLVIYGKNNRPSGIPDRFSGSSSGNTASLTIT
GAQAEDEADYYCNSSVYTMPPVVFGGGTKLTVLG
Amino acid sequence of the F16 single chain Fv (ScFv)
In BOLD UNDERLINED is the ten amino acid linker between VH and VL of the single
chain Fv
(SEQ ID NO: 23)
EVQLLESGGGLVQPGGSLRLSCAASGFTFSRYGMSWVRQAPGKGLEWVSAISGSGGSTYYADSVKGRFTISRDNS
KNTLYLQMNSLRAEDTAVYYCAKAHNAFDYWGQGTLVTVSRGGGSGGGSGGSSELTQDPAVSVALGQTVRITCQG
DSLRSYYASWYQQKPGQAPVLVIYGKNNRPSGIPDRFSGSSSGNTASLTITGAQAEDEADYYCNSSVYTMPPVVF
GGGTKLTVLG
Amino acid sequence of F16 diabody (Db)
In BOLD UNDERLINED is the five amino acid linker between VH and VL of the diabody
(SEQ ID NO: 24)
EVQLLESGGGLVQPGGSLRLSCAASGFTFSRYGMSWVRQAPGKGLEWVSAISGSGGSTYYADSVKGRFTISRDNS
KNTLYLQMNSLRAEDTAVYYCAKAHNAFDYWGQGTLVTVSRGGSGGSSELTQDPAVSVALGQTVRITCQGDSLRS
YYASWYQQKPGQAPVLVIYGKNNRPSGIPDRFSGSSSGNTASLTITGAQAEDEADYYCNSSVYTMPPVVFGGGTK
LTVLG
Amino acid sequences of F16 CDR's
F16 CDR1 VH
(SEQ ID NO: 25)
RYGMS
F16 CDR2 VH
(SEQ ID NO: 26)
AISGSGGSTYYADSVKG
F16 CDR3 VH
(SEQ ID NO: 27)
AHNAFDY
F16 CDR1 VL
(SEQ ID NO: 28)
QGDSLRSYYAS
F16 CDR2 VL
(SEQ ID NO: 29)
GKNNRPS
F16 CDR3 VL
(SEQ ID NO: 30)
NSSVYTMPPVV
Amino acid sequence of human IL4
In BOLD UNDERLINED is the amino acid that may be mutated to prevent glycosylation
(SEQ ID NO: 31)
HKCDITLQEIIKTLNSLTEQKTLCTELTVTDIFAASKNTTEKETFCRAATVLRQFYSHHEKDTRCLGATAQQFHR
HKQLIRFLKRLDRNLWGLAGLNSCPVKEANQSTLENFLERLKTIMREKYSKCSS
Amino acid sequence of non-glycosylated human IL4
In BOLD UNDERLINED is the amino acid that has been mutated to prevent glycosylation
(SEQ ID NO: 32)
HKCDITLQEIIKTLNSLTEQKTLCTELTVTDIFAASKQTTEKETFCRAATVLRQFYSHHEKDTRCLGATAQQFHR
HKQLIRFLKRLDRNLWGLAGLNSCPVKEANQSTLENFLERLKTIMREKYSKCSS
Amino acid sequence of a linker conjugating a specific binding member to human IL4
(SEQ ID NO: 33)
SSSSGSSSSGSSSSG
Amino acid sequence of a linker conjugating a specific binding member to human IL4
(SEQ ID NO: 34)
GGGGSGGGGSGGGGS
Amino acid sequence of scFvF8-humanIL4-scFvF8
The sequence below shows (in order) the sequence encoding: (i) the first
F8 VH domain [italics]; (ii) a 14 amino acid linker [bold and underlined];
(iii) the first F8 VL domain; (iv) a 15 amino acid linker [bold] (v) hIL4
[underlined] (vi) a 15 amino acid linker [bold] (vii) the second F8 VH
domain [italics]; (viii) a 14 amino acid linker [bold and underlined]; and
(ix) the second F8 VL domain
(SEQ ID NO: 35)
EVQLLESGGGLVQPGGSLRLSCAASGFTFSLFTMSWVRQAPGKGLEWVSAISGSGGSTYYADSVKGRFTISRDNS
KNTLYLQMNSLRAEDTAVYYCAKSTHLYLFDYWGQGTLVTVSSGGGGSGGGGSGGGGEIVLTQSPGTLSLSPGER
ATLSCRASQSVSMPFLAWYQQKPGQAPRLLIYGASSRATGIPDRFSGSGSGTDFTLTISRLEPEDFAVYYCQQMR
GRPPTFGQGTKVEIKSSSSGSSSSGSSSSGHKCDITLQEIIKTLNSLTEQKTLCTELTVTDIFAASKNTTEKETF
CRAATVLRQFYSHHEKDTRCLGATAQQFHRHKQLIRFLKRLDRNLWGLAGLNSCPVKEANQSTLENFLERLKTIM
REKYSKCSSSSSSGSSSSGSSSSGEVQLLESGGGLVQPGGSLRLSCAASGFTFSLFTMSWVRQAPGKGLEWVSAI
SGSGGSTYYADSVKGRFTISRDNSKNTLYLQMNSLRAEDTAVYYCAKSTHLYLFDYWGQGTLVTVSSGGGGSGGG
GSGGGGEIVLTQSPGTLSLSPGERATLSCRASQSVSMPFLAWYQQKPGQAPRLLIYGASSRATGIPDRFSGSGSG
TDFTLTISRLEPEDFAVYYCQQMRGRPPTFGQGTKVEIK
Amino acid sequence of scFvF8-non-glycosylated human IL4-scFvF8
The sequence below shows (in order) the sequence encoding: (i) the first
F8 VH domain [italics]; (ii) a 14 amino acid linker [bold and underlined];
(iii) the first F8 VL domain; (iv) a 15 amino acid linker [bold] (v) hIL4
[underlined] (vi) a 15 amino acid linker [bold] (vii) the second F8 VH
domain [italics]; (viii) a 14 amino acid linker [bold and underlined]; and
(ix) the second F8 VL domain
(SEQ ID NO: 36)
EVQLLESGGGLVQPGGSLRLSCAASGFTFSLFTMSWVRQAPGKGLEWVSAISGSGGSTYYADSVKGRFTISRDNS
KNTLYLQMNSLRAEDTAVYYCAKSTHLYLFDYWGQGTLVTVSSGGGGSGGGGSGGGGEIVLTQSPGTLSLSPGER
ATLSCRASQSVSMPFLAWYQQKPGQAPRLLIYGASSRATGIPDRFSGSGSGTDFTLTISRLEPEDFAVYYCQQMR
GRPPTFGQGTKVEIKSSSSGSSSSGSSSSGHKCDITLQEIIKTLNSLTEQKTLCTELTVTDIFAASKQTTEKETF
CRAATVLRQFYSHHEKDTRCLGATAQQFHRHKQLIRFLKRLDRNLWGLAGLNSCPVKEANQSTLENFLERLKTIM
REKYSKCSSSSSSGSSSSGSSSSGEVQLLESGGGLVQPGGSLRLSCAASGFTFSLFTMSWVRQAPGKGLEWVSAI
SGSGGSTYYADSVKGRFTISRDNSKNTLYLQMNSLRAEDTAVYYCAKSTHLYLFDYWGQGTLVTVSSGGGGSGGG
GSGGGGEIVLTQSPGTLSLSPGERATLSCRASQSVSMPFLAWYQQKPGQAPRLLIYGASSRATGIPDRFSGSGSG
TDFTLTISRLEPEDFAVYYCQQMRGRPPTFGQGTKVEIK
Amino acid sequence encoding the [human]IL4-ScDbF8 conjugate
The below sequence shows (in order) the sequence encoding: (i) hIL4
[underlined], (ii) a 15 amino acid linker [bold]; (iii) the first F8 VH
domain [italics]; (iv) a 5 amino acid linker [bold and underlined]; (v) the
first F8 VL domain; (vi) a 15 amino acid linker [bold] (vii) the second F8
VH domain [italics]; (viii) a 5 amino acid linker [bold and underlined];
and (ix) the second F8 VL domain
hIL4-15AA Linker-F8V_{H}-5AA Linker-F8V_{L}-15AA Linker--F8V_{H}-5AA Linker-F8V_{L}
(SEQ ID NO: 37)
HKCDITLQEIIKTLNSLTEQKTLCTELTVTDIFAASKNTTEKETFCRAATVLRQFYSHHEKDTRCLGATAQQFHR
HKQLIRFLKRLDRNLWGLAGLNSCPVKEANQSTLENFLERLKTIMREKYSKCSSGGGGSGGGGSGGGGSEVQLLE
SGGGLVQPGGSLRLSCAASGFTFSLFTMSWVRQAPGKGLEWVSAISGSGGSTYYADSVKGRFTISRDNSKNTLYL
QMNSLRAEDTAVYYCAKSTHLYLFDYWGQGTLVTVSSGGSGGEIVLTQSPGTLSLSPGERATLSCRASQSVSMPF
LAWYQQKPGQAPRLLIYGASSRATGIPDRFSGSGSGTDFTLTISRLEPEDFAVYYCQQMRGRPPTFGQGTKVEIK
SSSSGSSSSGSSSSGEVQLLESGGGLVQPGGSLRLSCAASGFTFSLFTMSWVRQAPGKGLEWVSAISGSGGSTYY
ADSVKGRFTISRDNSKNTLYLQMNSLRAEDTAVYYCAKSTHLYLFDYWGQGTLVTVSSGGSGGEIVLTQSPGTLS
LSPGERATLSCRASQSVSMPFLAWYQQKPGQAPRLLIYGASSRATGIPDRFSGSGSGTDFTLTISRLEPEDFAVY
YCQQMRGRPPTFGQGTKVEIK
Amino acid sequence encoding the ScDbF8-[human]IL4 conjugate
The below sequence shows (in order) the sequence encoding: (i) the first F8
VH domain [italics]; (ii) a 5 amino acid linker [bold and underlined]; (iii)
the first F8 VL domain; (iv) a 15 amino acid linker [bold] (v) the second
F8 VH domain [italics]; (vi) a 5 amino acid linker [bold and underlined];
(vii) the second F8 VL domain (viii) a 15 amino acid linker [bold] and (ix)
hIL4 [underlined]
F8V_{H}-5AA Linker-F8V_{L}-15AA Linker--F8V_{H}-5AA Linker-F8V_{L}-15AA Linker-hIL4
(SEQ ID NO: 38)
EVQLLESGGGLVQPGGSLRLSCAASGFTFSLFTMSWVRQAPGKGLEWVSAISGSGGSTYYADSVKGRFTISRDNS
KNTLYLQMNSLRAEDTAVYYCAKSTHLYLFDYWGQGTLVTVSSGGSGGEIVLTQSPGTLSLSPGERATLSCRASQ
SVSMPFLAWYQQKPGQAPRLLIYGASSRATGIPDRFSGSGSGTDFTLTISRLEPEDFAVYYCQQMRGRPPTFGQG
TKVEIKSSSSGSSSSGSSSSGEVQLLESGGGLVQPGGSLRLSCAASGFTFSLFTMSWVRQAPGKGLEWVSAISGS
GGSTYYADSVKGRFTISRDNSKNTLYLQMNSLRAEDTAVYYCAKSTHLYLFDYWGQGTLVTVSSGGSGGEIVLTQ
SPGTLSLSPGERATLSCRASQSVSMPFLAWYQQKPGQAPRLLIYGASSRATGIPDRFSGSGSGTDFTLTISRLEP
EDFAVYYCQQMRGRPPTFGQGTKVEIKGGGGSGGGGSGGGGSHKCDITLQEIIKTLNSLTEQKTLCTELTVTDIF
AASKNTTEKETFCRAATVLRQFYSHHEKDTRCLGATAQQFHRHKQLIRFLKRLDRNLWGLAGLNSCPVKEANQST
LENFLERLKTIMREKYSKCSS
Amino acid sequence of the linker between the VH and VL domains in the
diabody format and the single chain diabody format
(SEQ ID NO: 39)
GGSGG
Amino acid sequence of the linker between VH and VL domain in the ScFv format
(SEQ ID NO: 40)
GGGGSGGGGSGGGG
Nucleotide sequence encoding the hIL4-ScDbF8 conjugate
The below sequence shows (in order) the sequence encoding: (i) hIL4
[underlined], (ii) a 15 amino acid linker [bold]; (iii) the first F8 VH
domain [italics]; (iv) a 5 amino acid linker [bold and underlined]; (v) the
first F8 VL domain; (vi) a 15 amino acid linker [bold] (vii) the second F8
VH domain [italics]; (viii) a 5 amino acid linker [bold and underlined];
(ix) the second F8 VL domain and (x) the stop codon [bold]
hIL4-15AA Linker-F8V_{H}-5AA Linker-F8V_{L}-15AA Linker--F8V_{H}-5AA Linker-F8V_{L}
(SEQ ID NO: 41)
CACAAGTGCGATATCACCTTACAGGAGATCATCAAAACTTTGAACAGCCTCACAGAGCAGAAGACTCTGTGCACC
GAGTTGACCGTAACAGACATCTTTGCTGCCTCCAAGAACACAACTGAGAAGGAAACCTTCTGCAGGGCTGCGACT
GTGCTCCGGCAGTTCTACAGCCACCATGAGAAGGACACTCGCTGCCTGGGTGCGACTGCACAGCAGTTCCACAGG
CACAAGCAGCTGATCCGATTCCTGAAACGGCTCGACAGGAACCTCTGGGGCCTGGCGGGCTTGAATTCCTGTCCT
GTGAAGGAAGCCAACCAGAGTACGTTGGAAAACTTCTTGGAAAGGCTAAAGACGATCATGAGAGAGAAATATTCA
AAGTGTTCGAGCGGTGGAGGCGGTTCCGGAGGAGGTGGCTCTGGCGGTGGCGGATCAGAGGTGCAGCTGTTGGAG
TCTGGGGGAGGCTTGGTACAGCCTGGGGGGTCCCTGAGACTCTCCTGTGCAGCCTCTGGATTCACCTTTAGCCTG
TTTACGATGAGCTGGGTCCGCCAGGCTCCAGGGAAGGGGCTGGAGTGGGTCTCAGCTATTAGTGGTAGTGGTGGT
AGCACATACTACGCAGACTCCGTGAAGGGCCGGTTCACCATCTCCAGAGACAATTCCAAGAACACGCTGTATCTG
CAAATGAACAGCCTGAGAGCCGAGGACACGGCCGTATATTACTGTGCGAAAAGTACTCATTTGTATCTTTTTGAC
TACTGGGGCCAGGGAACCCTGGTCACCGTCTCGAGTGGCGGTAGCGGAGGGGAAATTGTGTTGACGCAGTCTCCA
GGCACCCTGTCTTTGTCTCCAGGGGAAAGAGCCACCCTCTCCTGCAGGGCCAGTCAGAGTGTTAGCATGCCGTTT
TTAGCCTGGTACCAGCAGAAACCTGGCCAGGCTCCCAGGCTCCTCATCTATGGTGCATCCAGCAGGGCCACTGGC
ATCCCAGACAGGTTCAGTGGCAGTGGGTCTGGGACAGACTTCACTCTCACCATCAGCAGACTGGAGCCTGAAGAT
TTTGCAGTGTATTACTGTCAGCAGATGCGTGGTCGGCCGCCGACGTTCGGCCAAGGGACCAAGGTGGAAATCAAA
TCTTCCTCAAGCGGATCCAGCTCTTCCGGCTCATCGTCCAGCGGCGAGGTGCAGCTGTTGGAGTCTGGGGGAGGC
TTGGTACAGCCTGGGGGGTCCCTGAGACTCTCCTGTGCAGCCTCTGGATTCACCTTTAGCCTGTTTACGATGAGC
TGGGTCCGCCAGGCTCCAGGGAAGGGGCTGGAGTGGGTCTCAGCTATTAGTGGTAGTGGTGGTAGCACATACTAC
GCAGACTCCGTGAAGGGCCGGTTCACCATCTCCAGAGACAATTCCAAGAACACGCTGTATCTGCAAATGAACAGC
CTGAGAGCCGAGGACACGGCCGTATATTACTGTGCGAAAAGTACTCATTTGTATCTTTTTGACTACTGGGGCCAG
GGAACCCTGGTCACCGTCTCGAGTGGCGGTAGCGGAGGGGAAATTGTGTTGACGCAGTCTCCAGGCACCCTGTCT
TTGTCTCCAGGGGAAAGAGCCACCCTCTCCTGCAGGGCCAGTCAGAGTGTTAGCATGCCGTTTTTAGCCTGGTAC
CAGCAGAAACCTGGCCAGGCTCCCAGGCTCCTCATCTATGGTGCATCCAGCAGGGCCACTGGCATCCCAGACAGG
TTCAGTGGCAGTGGGTCTGGGACAGACTTCACTCTCACCATCAGCAGACTGGAGCCTGAAGATTTTGCAGTGTAT
TACTGTCAGCAGATGCGTGGTCGGCCGCCGACGTTCGGCCAAGGGACCAAGGTGGAAATCAAATGA
Nucleotide sequence encoding the ScDbF8-hIL4 conjugate
The below sequence shows (in order) the sequence encoding: (i) the first F8
VH domain [italics]; (ii) a 5 amino acid linker [bold and underlined]; (iii)
the first F8 VL domain; (iv) a 15 amino acid linker [bold] (v) the second F8
VH domain [italics]; (vi) a 5 amino acid linker [bold and underlined]; (vii)
the second F8 VL domain (viii) a 15 amino acid linker [bold] (ix) hIL4
[underlined] and (x) the stop codon [bold]
F8V_{H}-5AA Linker-F8V_{L}-15AA Linker--F8V_{H}-5AA Linker-F8V_{L}-15AA Linker-hIL4
(SEQ ID NO: 42)
GAGGTGCAGCTGTTGGAGTCTGGGGGAGGCTTGGTACAGCCTGGGGGGTCCCTGAGACTCTCCTGTGCAGCCTCT
GGATTCACCTTTAGCCTGTTTACGATGAGCTGGGTCCGCCAGGCTCCAGGGAAGGGGCTGGAGTGGGTCTCAGCT
ATTAGTGGTAGTGGTGGTAGCACATACTACGCAGACTCCGTGAAGGGCCGGTTCACCATCTCCAGAGACAATTCC
AAGAACACGCTGTATCTGCAAATGAACAGCCTGAGAGCCGAGGACACGGCCGTATATTACTGTGCGAAAAGTACT
CATTTGTATCTTTTTGACTACTGGGGCCAGGGAACCCTGGTCACCGTCTCGAGTGGCGGTAGCGGAGGGGAAATT
GTGTTGACGCAGTCTCCAGGCACCCTGTCTTTGTCTCCAGGGGAAAGAGCCACCCTCTCCTGCAGGGCCAGTCAG
AGTGTTAGCATGCCGTTTTTAGCCTGGTACCAGCAGAAACCTGGCCAGGCTCCCAGGCTCCTCATCTATGGTGCA
TCCAGCAGGGCCACTGGCATCCCAGACAGGTTCAGTGGCAGTGGGTCTGGGACAGACTTCACTCTCACCATCAGC
AGACTGGAGCCTGAAGATTTTGCAGTGTATTACTGTCAGCAGATGCGTGGTCGGCCGCCGACGTTCGGCCAAGGG
ACCAAGGTGGAAATCAAATCTTCCTCAAGCGGATCCAGCTCTTCCGGCTCATCGTCCAGCGGCGAGGTGCAGCTG
TTGGAGTCTGGGGGAGGCTTGGTACAGCCTGGGGGGTCCCTGAGACTCTCCTGTGCAGCCTCTGGATTCACCTTT
AGCCTGTTTACGATGAGCTGGGTCCGCCAGGCTCCAGGGAAGGGGCTGGAGTGGGTCTCAGCTATTAGTGGTAGT
GGTGGTAGCACATACTACGCAGACTCCGTGAAGGGCCGGTTCACCATCTCCAGAGACAATTCCAAGAACACGCTG
TATCTGCAAATGAACAGCCTGAGAGCCGAGGACACGGCCGTATATTACTGTGCGAAAAGTACTCATTTGTATCTT
TTTGACTACTGGGGCCAGGGAACCCTGGTCACCGTCTCGAGTGGCGGTAGCGGAGGGGAAATTGTGTTGACGCAG
TCTCCAGGCACCCTGTCTTTGTCTCCAGGGGAAAGAGCCACCCTCTCCTGCAGGGCCAGTCAGAGTGTTAGCATG
CCGTTTTTAGCCTGGTACCAGCAGAAACCTGGCCAGGCTCCCAGGCTCCTCATCTATGGTGCATCCAGCAGGGCC
ACTGGCATCCCAGACAGGTTCAGTGGCAGTGGGTCTGGGACAGACTTCACTCTCACCATCAGCAGACTGGAGCCT
GAAGATTTTGCAGTGTATTACTGTCAGCAGATGCGTGGTCGGCCGCCGACGTTCGGCCAAGGGACCAAGGTGGAA
ATCAAAGGTGGAGGCGGTTCCGGAGGAGGTGGCTCTGGCGGTGGCGGATCACACAAGTGCGATATCACCTTACAG
GAGATCATCAAAACTTTGAACAGCCTCACAGAGCAGAAGACTCTGTGCACCGAGTTGACCGTAACAGACATCTTT
GCTGCCTCCAAGAACACAACTGAGAAGGAAACCTTCTGCAGGGCTGCGACTGTGCTCCGGCAGTTCTACAGCCAC
CATGAGAAGGACACTCGCTGCCTGGGTGCGACTGCACAGCAGTTCCACAGGCACAAGCAGCTGATCCGATTCCTG
AAACGGCTCGACAGGAACCTCTGGGGCCTGGCGGGCTTGAATTCCTGTCCTGTGAAGGAAGCCAACCAGAGTACG
TTGGAAAACTTCTTGGAAAGGCTAAAGACGATCATGAGAGAGAAATATTCAAAGTGTTCGAGCTGA
Nucleotide sequence encoding the scFvF8-hIL4-scFvF8 conjugate
The below sequence shows (in order) the sequence encoding: (i) the first F8
VH domain [italics]; (ii) a 14 amino acid linker [bold and underlined];
(iii) the first F8 VL domain; (iv) a 15 amino acid linker [bold] (v) hIL4
[underlined] (vi) a 15 amino acid linker [bold] (vii) the second F8 VH
domain [italics]; (viii) a 14 amino acid linker [bold and underlined]; (ix)
the second F8 VL domain and (x) the stop codon [bold]
F8V_{H}-15AA Linker-F8V_{L}-15AA Linker-hIL4-15AA Linker-F8V_{H}-15AA Linker-F8V_{L}
(SEQ ID NO: 43)
GAGGTGCAGCTGTTGGAGTCTGGGGGAGGCTTGGTACAGCCTGGGGGGTCCCTGAGACTCTCCTGTGCAGCCTCT
GGATTCACCTTTAGCCTGTTTACGATGAGCTGGGTCCGCCAGGCTCCAGGGAAGGGGCTGGAGTGGGTCTCAGCT
ATTAGTGGTAGTGGTGGTAGCACATACTACGCAGACTCCGTGAAGGGCCGGTTCACCATCTCCAGAGACAATTCC
AAGAACACGCTGTATCTGCAAATGAACAGCCTGAGAGCCGAGGACACGGCCGTATATTACTGTGCGAAAAGTACT
CATTTGTATCTTTTTGACTACTGGGGCCAGGGAACCCTGGTCACCGTCTCGAGTGGTGGAGGCGGTTCAGGCGGA
GGTGGCTCTGGCGGTGGCGGAGAAATTGTGTTGACGCAGTCTCCAGGCACCCTGTCTTTGTCTCCAGGGGAAAGA
GCCACCCTCTCCTGCAGGGCCAGTCAGAGTGTTAGCATGCCGTTTTTAGCCTGGTACCAGCAGAAACCTGGCCAG
GCTCCCAGGCTCCTCATCTATGGTGCATCCAGCAGGGCCACTGGCATCCCAGACAGGTTCAGTGGCAGTGGGTCT
GGGACAGACTTCACTCTCACCATCAGCAGACTGGAGCCTGAAGATTTTGCAGTGTATTACTGTCAGCAGATGCGT
GGTCGGCCGCCGACGTTCGGCCAAGGGACCAAGGTGGAAATCAAATCTTCCTCAAGCGGATCCAGCTCTTCCGGC
TCATCGTCCAGCGGCCACAAGTGCGATATCACCTTACAGGAGATCATCAAAACTTTGAACAGCCTCACAGAGCAG
AAGACTCTGTGCACCGAGTTGACCGTAACAGACATCTTTGCTGCCTCCAAGAACACAACTGAGAAGGAAACCTTC
TGCAGGGCTGCGACTGTGCTCCGGCAGTTCTACAGCCACCATGAGAAGGACACTCGCTGCCTGGGTGCGACTGCA
CAGCAGTTCCACAGGCACAAGCAGCTGATCCGATTCCTGAAACGGCTCGACAGGAACCTCTGGGGCCTGGCGGGC
TTGAATTCCTGTCCTGTGAAGGAAGCCAACCAGAGTACGTTGGAAAACTTCTTGGAAAGGCTAAAGACGATCATG
AGAGAGAAATATTCAAAGTGTTCGAGCTCATCCTCTAGTGGTAGCTCTTCATCCGGAAGCTCCTCGTCTGGTGAG
GTGCAGCTGTTGGAGTCTGGGGGAGGCTTGGTACAGCCTGGGGGGTCCCTGAGACTCTCCTGTGCAGCCTCTGGA
TTCACCTTTAGCCTGTTTACGATGAGCTGGGTCCGCCAGGCTCCAGGGAAGGGGCTGGAGTGGGTCTCAGCTATT
AGTGGTAGTGGTGGTAGCACATACTACGCAGACTCCGTGAAGGGCCGGTTCACCATCTCCAGAGACAATTCCAAG
AACACGCTGTATCTGCAAATGAACAGCCTGAGAGCCGAGGACACGGCCGTATATTACTGTGCGAAAAGTACTCAT
TTGTATCTTTTTGACTACTGGGGCCAGGGAACCCTGGTCACCGTCTCGAGTGGTGGAGGCGGTTCAGGCGGAGGT
GGCTCTGGCGGTGGCGGAGAAATTGTGTTGACGCAGTCTCCAGGCACCCTGTCTTTGTCTCCAGGGGAAAGAGCC
ACCCTCTCCTGCAGGGCCAGTCAGAGTGTTAGCATGCCGTTTTTAGCCTGGTACCAGCAGAAACCTGGCCAGGCT
CCCAGGCTCCTCATCTATGGTGCATCCAGCAGGGCCACTGGCATCCCAGACAGGTTCAGTGGCAGTGGGTCTGGG
ACAGACTTCACTCTCACCATCAGCAGACTGGAGCCTGAAGATTTTGCAGTGTATTACTGTCAGCAGATGCGTGGT
CGGCCGCCGACGTTCGGCCAAGGGACCAAGGTGGAAATCAAATAG
Amino acid sequence of the linker between VH and VL domain in the ScFv format
(SEQ ID NO: 44)
GDGSSGGSGGAS
Amino acid sequence of the linker between VH and VL domain in the ScFv format
(SEQ ID NO: 45)
GGGSGGGSGG
Amino acid sequence of murine IL4
(SEQ ID NO: 46)
HIHGCDKNHLREIIGILNEVTGEGTPCTEMDVPNVLTATKNTTESELVCRASKVLRIFYLKHGKTPCLKKNSSVL
MELQRLFRAFRCLDSSISCTMNESKSTSLKDFLESLKSIMQMDYS

1. A conjugate comprising interleukin-4 (IL4) and two antibody molecules which bind an extra-cellular matrix component associated with neoplastic growth, angiogenesis, and/or tissue remodelling,
wherein the N terminus of IL4 is conjugated to a first antibody molecule and the C terminus of IL4 is conjugated to a second antibody molecule.

2. A conjugate according to claim 1 wherein the antibody molecules are conjugated to the N and C termini of the IL4 via amino acid linkers.

3. A conjugate according to claim 1 wherein the extra-cellular matrix component is fibronectin.

4-6. (canceled)

7. A conjugate according to claim 3 wherein the antibody molecules bind to the Extra Domain-A (ED-A) of fibronectin.

8. A conjugate according to claim 7 wherein the antibody molecules comprise an antigen binding site having the complementarity determining regions (CDRs) of antibody F8 set forth in SEQ ID NOs 5-10.

9. A conjugate according to claim 7 wherein the antibody molecules comprises a VH domain and a VL domain of F8 of SEQ ID NOS: 1 and 2 respectively.

10-12. (canceled)

13. A conjugate according to claim 7 wherein the antibody molecules comprise the amino acid sequence of F8 set forth in SEQ ID NO: 3.

14. A conjugate according to claim 9 wherein the antibody molecules bind to the Extra Domain-B (ED-B) of fibronectin.

15. A conjugate according to claim 14 wherein the antibody molecules comprise an antigen binding site having the complementarity determining regions (CDRs) of antibody L19 set forth in SEQ ID NOs 15-20.

16. A conjugate according to claim 14 wherein the antibody molecules comprise VH domains and VL domains of antibody L19 VH set forth in SEQ ID NOs: 11 and 12.

17-19. (canceled)

20. A conjugate according to claim 14 wherein the antibody molecules comprise a L19 amino acid sequence of SEQ ID NO: 13.

21. A conjugate according to claim 1 wherein the extra-cellular matrix component is the A1 domain of Tenascin C.

22. A conjugate according to claim 21 wherein the antibody molecules comprise an antigen binding site having the complementarity determining regions (CDRs) of antibody F16 set forth in SEQ ID NOs 25-30.

23. (canceled)

24. (canceled)

25. A conjugate according to claim 21 wherein the antibody molecules comprise VH and VL domains of antibody F16 set forth in SEQ ID NOs 21 and 22.

26. (canceled)

27. A conjugate according to claim 21 wherein the antibody molecule comprises the F16 amino acid sequence of SEQ ID NO: 23

28-30. (canceled)

31. A conjugate according to 7 comprising the amino acid sequence of SEQ ID NOs 35-36.

32. A nucleic acid molecule encoding a conjugate according to claim 1.

33. (canceled)

34. An expression vector comprising the nucleic acid of claim 32.

35. A host cell comprising the vector of claim 34.

36-42. (canceled)

43. A method of treating of a disease characterised by expression of the extra-cellular matrix component associated with neoplastic growth, angiogenesis, and/or tissue remodelling in a patient, the method comprising administering a therapeutically effective amount of a conjugate according to claim 1 to the patient.

44. A method of delivering IL4 to sites of disease characterised by expression of an extra-cellular matrix component associated with neoplastic growth, angiogenesis, and/or tissue remodelling in a patient comprising administering the conjugate according to claim 1 to the patient.

45-50. (canceled)

51. A method according to claim 43 wherein the disease is characterised by neoplastic growth and/or angiogenesis.

52. A method according to claim 46 wherein the disease is cancer.

53. A method according to claim 43 wherein the disease is an inflammatory autoimmune disease.

54. A method according to claim 44 wherein the disease is characterised by neoplastic growth and/or angiogenesis.

55. A method according to claim 51 wherein the disease is cancer.

56. A method according to claim 44 wherein the disease is an inflammatory autoimmune disease.


SEQUENCE LISTING (46 sequences)

SEQ ID NO: 1 - protein, 118 aa, Artificial Sequence
  Amino acid sequence of F8 VH
EVQLLESGGGLVQPGGSLRLSCAASGFTFSLFTMSWVRQAPGKGLEWVSAISGSGGSTYYADSVKGRFTISRDNSKNTLYLQMNSLRAEDTAVYYCAKSTHLYLFDYWGQGTLVTVSS

SEQ ID NO: 2 - protein, 108 aa, Artificial Sequence
  Amino acid sequence of F8 VL
EIVLTQSPGTLSLSPGERATLSCRASQSVSMPFLAWYQQKPGQAPRLLIYGASSRATGIPDRFSGSGSGTDFTLTISRLEPEDFAVYYCQQMRGRPPTFGQGTKVEIK

SEQ ID NO: 3 - protein, 240 aa, Artificial Sequence
  Amino acid sequence of the F8 single chain Fv (ScFv)
EVQLLESGGGLVQPGGSLRLSCAASGFTFSLFTMSWVRQAPGKGLEWVSAISGSGGSTYYADSVKGRFTISRDNSKNTLYLQMNSLRAEDTAVYYCAKSTHLYLFDYWGQGTLVTVSSGGGGSGGGGSGGGGEIVLTQSPGTLSLSPGERATLSCRASQSVSMPFLAWYQQKPGQAPRLLIYGASSRATGIPDRFSGSGSGTDFTLTISRLEPEDFAVYYCQQMRGRPPTFGQGTKVEIK

SEQ ID NO: 4 - protein, 231 aa, Artificial Sequence
  Amino acid sequence of F8 diabody (Db)
EVQLLESGGGLVQPGGSLRLSCAASGFTFSLFTMSWVRQAPGKGLEWVSAISGSGGSTYYADSVKGRFTISRDNSKNTLYLQMNSLRAEDTAVYYCAKSTHLYLFDYWGQGTLVTVSSGGSGGEIVLTQSPGTLSLSPGERATLSCRASQSVSMPFLAWYQQKPGQAPRLLIYGASSRATGIPDRFSGSGSGTDFTLTISRLEPEDFAVYYCQQMRGRPPTFGQGTKVEIK

SEQ ID NO: 5 - protein, 3 aa, Artificial Sequence
  F8 CDR1 VH
LFT

SEQ ID NO: 6 - protein, 6 aa, Artificial Sequence
  F8 CDR2 VH
SGSGGS

SEQ ID NO: 7 - protein, 6 aa, Artificial Sequence
  F8 CDR3 VH
STHLYL

SEQ ID NO: 8 - protein, 3 aa, Artificial Sequence
  F8 CDR1 VL
MPF

SEQ ID NO: 9 - protein, 7 aa, Artificial Sequence
  F8 CDR2 VL
GASSRAT

SEQ ID NO: 10 - protein, 6 aa, Artificial Sequence
  F8 CDR3 VL
MRGRPP

SEQ ID NO: 11 - protein, 116 aa, Artificial Sequence [sequence omitted]

SEQ ID NO: 12 - protein, 108 aa, Artificial Sequence [sequence omitted]

SEQ ID NO: 13 - protein, 236 aa, Artificial Sequence
  Amino acid sequence of the L19 single chain Fv (ScFv)
EVQLLESGGGLVQPGGSLRLSCAASGFTFSSFSMSWVRQAPGKGLEWVSSISGSSGTTYYADSVKGRFTISRDNSKNTLYLQMNSLRAEDTAVYYCAKPFPYFDYWGQGTLVTVSSGDGSSGGSGGASEIVLTQSPGTLSLSPGERATLSCRASQSVSSSFLAWYQQKPGQAPRLLIYYASSRATGIPDRFSGSGSGTDFTLTISRLEPEDFAVYYCQQTGRIPPTFGQGTKVEIK

SEQ ID NO: 14 - protein, 229 aa, Artificial Sequence
  Amino acid sequence of the L19 diabody (Db)
EVQLLESGGGLVQPGGSLRLSCAASGFTFSSFSMSWVRQAPGKGLEWVSSISGSSGTTYYADSVKGRFTISRDNSKNTLYLQMNSLRAEDTAVYYCAKPFPYFDYWGQGTLVTVSSGGSGGEIVLTQSPGTLSLSPGERATLSCRASQSVSSSFLAWYQQKPGQAPRLLIYYASSRATGIPDRFSGSGSGTDFTLTISRLEPEDFAVYYCQQTGRIPPTFGQGTKVEIK

SEQ ID NO: 15 - protein, 5 aa, Artificial Sequence
  L19 CDR1 VH
SFSMS

SEQ ID NO: 16 - protein, 17 aa, Artificial Sequence
  L19 CDR2 VH
SISGSSGTTYYADSVKG

SEQ ID NO: 17 - protein, 7 aa, Artificial Sequence
  L19 CDR3 VH
PFPYFDY

SEQ ID NO: 18 - protein, 12 aa, Artificial Sequence
  L19 CDR1 VL
RASQSVSSSFLA

SEQ ID NO: 19 - protein, 7 aa, Artificial Sequence
  L19 CDR2 VL
YASSRAT

SEQ ID NO: 20 - protein, 9 aa, Artificial Sequence
  L19 CDR3 VL
QQTGRIPPT

SEQ ID NO: 21 - protein, 116 aa, Artificial Sequence
  Amino acid sequence of F16 VH
EVQLLESGGGLVQPGGSLRLSCAASGFTFSRYGMSWVRQAPGKGLEWVSAISGSGGSTYYADSVKGRFTISRDNSKNTLYLQMNSLRAEDTAVYYCAKAHNAFDYWGQGTLVTVSR

SEQ ID NO: 22 - protein, 109 aa, Artificial Sequence
  Amino acid sequence of F16 VL
SSELTQDPAVSVALGQTVRITCQGDSLRSYYASWYQQKPGQAPVLVIYGKNNRPSGIPDRFSGSSSGNTASLTITGAQAEDEADYYCNSSVYTMPPVVFGGGTKLTVLG

SEQ ID NO: 23 - protein, 235 aa, Artificial Sequence
  Amino acid sequence of the F16 single chain Fv (ScFv)
EVQLLESGGGLVQPGGSLRLSCAASGFTFSRYGMSWVRQAPGKGLEWVSAISGSGGSTYYADSVKGRFTISRDNSKNTLYLQMNSLRAEDTAVYYCAKAHNAFDYWGQGTLVTVSRGGGSGGGSGGSSELTQDPAVSVALGQTVRITCQGDSLRSYYASWYQQKPGQAPVLVIYGKNNRPSGIPDRFSGSSSGNTASLTITGAQAEDEADYYCNSSVYTMPPVVFGGGTKLTVLG

SEQ ID NO: 24 - protein, 230 aa, Artificial Sequence
  Amino acid sequence of F16 diabody (Db)
EVQLLESGGGLVQPGGSLRLSCAASGFTFSRYGMSWVRQAPGKGLEWVSAISGSGGSTYYADSVKGRFTISRDNSKNTLYLQMNSLRAEDTAVYYCAKAHNAFDYWGQGTLVTVSRGGSGGSSELTQDPAVSVALGQTVRITCQGDSLRSYYASWYQQKPGQAPVLVIYGKNNRPSGIPDRFSGSSSGNTASLTITGAQAEDEADYYCNSSVYTMPPVVFGGGTKLTVLG

SEQ ID NO: 25 - protein, 5 aa, Artificial Sequence
  F16 CDR1 VH
RYGMS

SEQ ID NO: 26 - protein, 17 aa, Artificial Sequence
  F16 CDR2 VH
AISGSGGSTYYADSVKG

SEQ ID NO: 27 - protein, 7 aa, Artificial Sequence
  F16 CDR3 VH
AHNAFDY

SEQ ID NO: 28 - protein, 11 aa, Artificial Sequence
  F16 CDR1 VL
QGDSLRSYYAS

SEQ ID NO: 29 - protein, 7 aa, Artificial Sequence
  F16 CDR2 VL
GKNNRPS

SEQ ID NO: 30 - protein, 11 aa, Artificial Sequence
  F16 CDR3 VL
NSSVYTMPPVV

SEQ ID NO: 31 - protein, 129 aa, Artificial Sequence [sequence omitted]

SEQ ID NO: 32 - protein, 129 aa, Artificial Sequence
  Amino acid sequence of non-glycosylated human IL4
HKCDITLQEIIKTLNSLTEQKTLCTELTVTDIFAASKQTTEKETFCRAATVLRQFYSHHEKDTRCLGATAQQFHRHKQLIRFLKRLDRNLWGLAGLNSCPVKEANQSTLENFLERLKTIMREKYSKCSS

SEQ ID NO: 33 - protein, 15 aa, Artificial Sequence
  Amino acid sequence of a linker conjugating a specific binding member to human IL4
SSSSGSSSSGSSSSG

SEQ ID NO: 34 - protein, 15 aa, Artificial Sequence
  Amino acid sequence of a linker conjugating a specific binding member to human IL4
GGGGSGGGGSGGGGS

SEQ ID NO: 35 - protein, 639 aa, Artificial Sequence
  Amino acid sequence of scFvF8-humanIL4-scFvF8
EVQLLESGGGLVQPGGSLRLSCAASGFTFSLFTMSWVRQAPGKGLEWVSAISGSGGSTYYADSVKGRFTISRDNSKNTLYLQMNSLRAEDTAVYYCAKSTHLYLFDYWGQGTLVTVSSGGGGSGGGGSGGGGEIVLTQSPGTLSLSPGERATLSCRASQSVSMPFLAWYQQKPGQAPRLLIYGASSRATGIPDRFSGSGSGTDFTLTISRLEPEDFAVYYCQQMRGRPPTFGQGTKVEIKSSSSGSSSSGSSSSGHKCDITLQEIIKTLNSLTEQKTLCTELTVTDIFAASKNTTEKETFCRAATVLRQFYSHHEKDTRCLGATAQQFHRHKQLIRFLKRLDRNLWGLAGLNSCPVKEANQSTLENFLERLKTIMREKYSKCSSSSSSGSSSSGSSSSGEVQLLESGGGLVQPGGSLRLSCAASGFTFSLFTMSWVRQAPGKGLEWVSAISGSGGSTYYADSVKGRFTISRDNSKNTLYLQMNSLRAEDTAVYYCAKSTHLYLFDYWGQGTLVTVSSGGGGSGGGGSGGGGEIVLTQSPGTLSLSPGERATLSCRASQSVSMPFLAWYQQKPGQAPRLLIYGASSRATGIPDRFSGSGSGTDFTLTISRLEPEDFAVYYCQQMRGRPPTFGQGTKVEIK

SEQ ID NO: 36 - protein, 639 aa, Artificial Sequence
  Amino acid sequence of scFvF8-non-glycosylated human IL4-scFvF8
EVQLLESGGGLVQPGGSLRLSCAASGFTFSLFTMSWVRQAPGKGLEWVSAISGSGGSTYYADSVKGRFTISRDNSKNTLYLQMNSLRAEDTAVYYCAKSTHLYLFDYWGQGTLVTVSSGGGGSGGGGSGGGGEIVLTQSPGTLSLSPGERATLSCRASQSVSMPFLAWYQQKPGQAPRLLIYGASSRATGIPDRFSGSGSGTDFTLTISRLEPEDFAVYYCQQMRGRPPTFGQGTKVEIKSSSSGSSSSGSSSSGHKCDITLQEIIKTLNSLTEQKTLCTELTVTDIFAASKQTTEKETFCRAATVLRQFYSHHEKDTRCLGATAQQFHRHKQLIRFLKRLDRNLWGLAGLNSCPVKEANQSTLENFLERLKTIMREKYSKCSSSSSSGSSSSGSSSSGEVQLLESGGGLVQPGGSLRLSCAASGFTFSLFTMSWVRQAPGKGLEWVSAISGSGGSTYYADSVKGRFTISRDNSKNTLYLQMNSLRAEDTAVYYCAKSTHLYLFDYWGQGTLVTVSSGGGGSGGGGSGGGGEIVLTQSPGTLSLSPGERATLSCRASQSVSMPFLAWYQQKPGQAPRLLIYGASSRATGIPDRFSGSGSGTDFTLTISRLEPEDFAVYYCQQMRGRPPTFGQGTKVEIK

SEQ ID NO: 37 - protein, 621 aa, Artificial Sequence
  Amino acid sequence encoding the [human]IL4- ScDbF8 conjugate
HKCDITLQEIIKTLNSLTEQKTLCTELTVTDIFAASKNTTEKETFCRAATVLRQFYSHHEKDTRCLGATAQQFHRHKQLIRFLKRLDRNLWGLAGLNSCPVKEANQSTLENFLERLKTIMREKYSKCSSGGGGSGGGGSGGGGSEVQLLESGGGLVQPGGSLRLSCAASGFTFSLFTMSWVRQAPGKGLEWVSAISGSGGSTYYADSVKGRFTISRDNSKNTLYLQMNSLRAEDTAVYYCAKSTHLYLFDYWGQGTLVTVSSGGSGGEIVLTQSPGTLSLSPGERATLSCRASQSVSMPFLAWYQQKPGQAPRLLIYGASSRATGIPDRFSGSGSGTDFTLTISRLEPEDFAVYYCQQMRGRPPTFGQGTKVEIKSSSSGSSSSGSSSSGEVQLLESGGGLVQPGGSLRLSCAASGFTFSLFTMSWVRQAPGKGLEWVSAISGSGGSTYYADSVKGRFTISRDNSKNTLYLQMNSLRAEDTAVYYCAKSTHLYLFDYWGQGTLVTVSSGGSGGEIVLTQSPGTLSLSPGERATLSCRASQSVSMPFLAWYQQKPGQAPRLLIYGASSRATGIPDRFSGSGSGTDFTLTISRLEPEDFAVYYCQQMRGRPPTFGQGTKVEIK

SEQ ID NO: 38 - protein, 621 aa, Artificial Sequence
  Amino acid sequence encoding the ScDbF8- [human]IL4 conjugate
EVQLLESGGGLVQPGGSLRLSCAASGFTFSLFTMSWVRQAPGKGLEWVSAISGSGGSTYYADSVKGRFTISRDNSKNTLYLQMNSLRAEDTAVYYCAKSTHLYLFDYWGQGTLVTVSSGGSGGEIVLTQSPGTLSLSPGERATLSCRASQSVSMPFLAWYQQKPGQAPRLLIYGASSRATGIPDRFSGSGSGTDFTLTISRLEPEDFAVYYCQQMRGRPPTFGQGTKVEIKSSSSGSSSSGSSSSGEVQLLESGGGLVQPGGSLRLSCAASGFTFSLFTMSWVRQAPGKGLEWVSAISGSGGSTYYADSVKGRFTISRDNSKNTLYLQMNSLRAEDTAVYYCAKSTHLYLFDYWGQGTLVTVSSGGSGGEIVLTQSPGTLSLSPGERATLSCRASQSVSMPFLAWYQQKPGQAPRLLIYGASSRATGIPDRFSGSGSGTDFTLTISRLEPEDFAVYYCQQMRGRPPTFGQGTKVEIKGGGGSGGGGSGGGGSHKCDITLQEIIKTLNSLTEQKTLCTELTVTDIFAASKNTTEKETFCRAATVLRQFYSHHEKDTRCLGATAQQFHRHKQLIRFLKRLDRNLWGLAGLNSCPVKEANQSTLENFLERLKTIMREKYSKCSS

SEQ ID NO: 39 - protein, 6 aa, Artificial Sequence
  Amino acid sequence of the linker between the VH and VL domains in the diabody format and the single chain diabody format
GGSSGG

SEQ ID NO: 40 - protein, 14 aa, Artificial Sequence
  Amino acid sequence of the linker between VH and VL domain in the ScFv format
GGGGSGGGGSGGGG

SEQ ID NO: 41 - DNA, 1866 bp, Artificial Sequence
  Nucleotide sequence encoding the hIL4-ScDbF8 conjugate
cacaagtgcgatatcaccttacaggagatcatcaaaactttgaacagcctcacagagcagaagactctgtgcaccgagttgaccgtaacagacatctttgctgcctccaagaacacaactgagaaggaaaccttctgcagggctgcgactgtgctccggcagttctacagccaccatgagaaggacactcgctgcctgggtgcgactgcacagcagttccacaggcacaagcagctgatccgattcctgaaacggctcgacaggaacctctggggcctggcgggcttgaattcctgtcctgtgaaggaagccaaccagagtacgttggaaaacttcttggaaaggctaaagacgatcatgagagagaaatattcaaagtgttcgagcggtggaggcggttccggaggaggtggctctggcggtggcggatcagaggtgcagctgttggagtctgggggaggcttggtacagcctggggggtccctgagactctcctgtgcagcctctggattcacctttagcctgtttacgatgagctgggtccgccaggctccagggaaggggctggagtgggtctcagctattagtggtagtggtggtagcacatactacgcagactccgtgaagggccggttcaccatctccagagacaattccaagaacacgctgtatctgcaaatgaacagcctgagagccgaggacacggccgtatattactgtgcgaaaagtactcatttgtatctttttgactactggggccagggaaccctggtcaccgtctcgagtggcggtagcggaggggaaattgtgttgacgcagtctccaggcaccctgtctttgtctccaggggaaagagccaccctctcctgcagggccagtcagagtgttagcatgccgtttttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcagatgcgtggtcggccgccgacgttcggccaagggaccaaggtggaaatcaaatcttcctcaagcggatccagctcttccggctcatcgtccagcggcgaggtgcagctgttggagtctgggggaggcttggtacagcctggggggtccctgagactctcctgtgcagcctctggattcacctttagcctgtttacgatgagctgggtccgccaggctccagggaaggggctggagtgggtctcagctattagtggtagtggtggtagcacatactacgcagactccgtgaagggccggttcaccatctccagagacaattccaagaacacgctgtatctgcaaatgaacagcctgagagccgaggacacggccgtatattactgtgcgaaaagtactcatttgtatctttttgactactggggccagggaaccctggtcaccgtctcgagtggcggtagcggaggggaaattgtgttgacgcagtctccaggcaccctgtctttgtctccaggggaaagagccaccctctcctgcagggccagtcagagtgttagcatgccgtttttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcagatgcgtggtcggccgccgacgttcggccaagggaccaaggtggaaatcaaatga

SEQ ID NO: 42 - DNA, 1866 bp, Artificial Sequence
  Nucleotide sequence encoding the ScDbF8-hIL4 conjugate
gaggtgcagctgttggagtctgggggaggcttggtacagcctggggggtccctgagactctcctgtgcagcctctggattcacctttagcctgtttacgatgagctgggtccgccaggctccagggaaggggctggagtgggtctcagctattagtggtagtggtggtagcacatactacgcagactccgtgaagggccggttcaccatctccagagacaattccaagaacacgctgtatctgcaaatgaacagcctgagagccgaggacacggccgtatattactgtgcgaaaagtactcatttgtatctttttgactactggggccagggaaccctggtcaccgtctcgagtggcggtagcggaggggaaattgtgttgacgcagtctccaggcaccctgtctttgtctccaggggaaagagccaccctctcctgcagggccagtcagagtgttagcatgccgtttttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcagatgcgtggtcggccgccgacgttcggccaagggaccaaggtggaaatcaaatcttcctcaagcggatccagctcttccggctcatcgtccagcggcgaggtgcagctgttggagtctgggggaggcttggtacagcctggggggtccctgagactctcctgtgcagcctctggattcacctttagcctgtttacgatgagctgggtccgccaggctccagggaaggggctggagtgggtctcagctattagtggtagtggtggtagcacatactacgcagactccgtgaagggccggttcaccatctccagagacaattccaagaacacgctgtatctgcaaatgaacagcctgagagccgaggacacggccgtatattactgtgcgaaaagtactcatttgtatctttttgactactggggccagggaaccctggtcaccgtctcgagtggcggtagcggaggggaaattgtgttgacgcagtctccaggcaccctgtctttgtctccaggggaaagagccaccctctcctgcagggccagtcagagtgttagcatgccgtttttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcagatgcgtggtcggccgccgacgttcggccaagggaccaaggtggaaatcaaaggtggaggcggttccggaggaggtggctctggcggtggcggatcacacaagtgcgatatcaccttacaggagatcatcaaaactttgaacagcctcacagagcagaagactctgtgcaccgagttgaccgtaacagacatctttgctgcctccaagaacacaactgagaaggaaaccttctgcagggctgcgactgtgctccggcagttctacagccaccatgagaaggacactcgctgcctgggtgcgactgcacagcagttccacaggcacaagcagctgatccgattcctgaaacggctcgacaggaacctctggggcctggcgggcttgaattcctgtcctgtgaaggaagccaaccagagtacgttggaaaacttcttggaaaggctaaagacgatcatgagagagaaatattcaaagtgttcgagctga

SEQ ID NO: 43 - DNA, 1920 bp, Artificial Sequence
  Nucleotide sequence encoding the scFvF8-hIL4- scFvF8 conjugate
gaggtgcagctgttggagtctgggggaggcttggtacagcctggggggtccctgagactctcctgtgcagcctctggattcacctttagcctgtttacgatgagctgggtccgccaggctccagggaaggggctggagtgggtctcagctattagtggtagtggtggtagcacatactacgcagactccgtgaagggccggttcaccatctccagagacaattccaagaacacgctgtatctgcaaatgaacagcctgagagccgaggacacggccgtatattactgtgcgaaaagtactcatttgtatctttttgactactggggccagggaaccctggtcaccgtctcgagtggtggaggcggttcaggcggaggtggctctggcggtggcggagaaattgtgttgacgcagtctccaggcaccctgtctttgtctccaggggaaagagccaccctctcctgcagggccagtcagagtgttagcatgccgtttttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcagatgcgtggtcggccgccgacgttcggccaagggaccaaggtggaaatcaaatcttcctcaagcggatccagctcttccggctcatcgtccagcggccacaagtgcgatatcaccttacaggagatcatcaaaactttgaacagcctcacagagcagaagactctgtgcaccgagttgaccgtaacagacatctttgctgcctccaagaacacaactgagaaggaaaccttctgcagggctgcgactgtgctccggcagttctacagccaccatgagaaggacactcgctgcctgggtgcgactgcacagcagttccacaggcacaagcagctgatccgattcctgaaacggctcgacaggaacctctggggcctggcgggcttgaattcctgtcctgtgaaggaagccaaccagagtacgttggaaaacttcttggaaaggctaaagacgatcatgagagagaaatattcaaagtgttcgagctcatcctctagtggtagctcttcatccggaagctcctcgtctggtgaggtgcagctgttggagtctgggggaggcttggtacagcctggggggtccctgagactctcctgtgcagcctctggattcacctttagcctgtttacgatgagctgggtccgccaggctccagggaaggggctggagtgggtctcagctattagtggtagtggtggtagcacatactacgcagactccgtgaagggccggttcaccatctccagagacaattccaagaacacgctgtatctgcaaatgaacagcctgagagccgaggacacggccgtatattactgtgcgaaaagtactcatttgtatctttttgactactggggccagggaaccctggtcaccgtctcgagtggtggaggcggttcaggcggaggtggctctggcggtggcggagaaattgtgttgacgcagtctccaggcaccctgtctttgtctccaggggaaagagccaccctctcctgcagggccagtcagagtgttagcatgccgtttttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcagatgcgtggtcggccgccgacgttcggccaagggaccaaggtggaaatcaaatag

SEQ ID NO: 44 - protein, 12 aa, Artificial Sequence
  Amino acid sequence of the linker between VH and VL domain in the ScFv format
GDGSSGGSGGAS

SEQ ID NO: 45 - protein, 10 aa, Artificial Sequence
  Amino acid sequence of the linker between VH and VL domain in the ScFv format
GGGSGGGSGG

SEQ ID NO: 46 - protein, 120 aa, Artificial Sequence
  Amino acid sequence of murine IL4
HIHGCDKNHLREIIGILNEVTGEGTPCTEMDVPNVLTATKNTTESELVCRASKVLRIFYLKHGKTPCLKKNSSVLMELQRLFRAFRCLDSSISCTMNESKSTSLKDFLESLKSIMQMDYS